SEQUENCE LISTING (21 sequences)

SEQ ID NO: 1 - DNA, 24 bp, synthetic construct
  source 1..24: mol_type = other DNA
tataggggttagggtagggagggc

SEQ ID NO: 2 - DNA, 23 bp, synthetic construct
  source 1..23: mol_type = other DNA
caaccctacccgaaacgccaacg

SEQ ID NO: 3 - DNA, 26 bp, synthetic construct
  source 1..26: mol_type = other DNA
cctctacccgcgaaaacccgaacctc

SEQ ID NO: 4 - DNA, 23 bp, synthetic construct
  source 1..23: mol_type = other DNA
gagaggcgaagtcgcgagcgttc

SEQ ID NO: 5 - DNA, 25 bp, synthetic construct
  source 1..25: mol_type = other DNA
ctatcataactacgaccacccgacg

SEQ ID NO: 6 - DNA, 26 bp, synthetic construct
  source 1..26: mol_type = other DNA
cgggcggtttcgtcgggtggtcgtag

SEQ ID NO: 7 - DNA, 24 bp, synthetic construct
  source 1..24: mol_type = other DNA
aacactcgctcgctccctacccga

SEQ ID NO: 8 - DNA, 26 bp, synthetic construct
  source 1..26: mol_type = other DNA
taactcactcgatcctacgaacgacg

SEQ ID NO: 9 - DNA, 24 bp, synthetic construct
  source 1..24: mol_type = other DNA
aacactcgctcgctccctacccga

SEQ ID NO: 10 - DNA, 26 bp, synthetic construct
  source 1..26: mol_type = other DNA
ttcgagcgggtcgcgatatatttttc

SEQ ID NO: 11 - DNA, 26 bp, synthetic construct
  source 1..26: mol_type = other DNA
ctcgaaaaccctaaataacctctccg

SEQ ID NO: 12 - DNA, 23 bp, synthetic construct
  source 1..23: mol_type = other DNA
agtagcgcggggagaaggcggag

SEQ ID NO: 13 - DNA, 25 bp, synthetic construct
  source 1..25: mol_type = other DNA
attatttagtaggcgattcgtcgcg

SEQ ID NO: 14 - DNA, 24 bp, synthetic construct
  source 1..24: mol_type = other DNA
cgctacaaaccgtactactaaccg

SEQ ID NO: 15 - DNA, 22 bp, synthetic construct
  source 1..22: mol_type = other DNA
ctacgaaccgcgacgaatcgcc

SEQ ID NO: 16 - DNA, 25 bp, synthetic construct
  source 1..25: mol_type = other DNA
gtcgtttgtttttcgttttaggttc

SEQ ID NO: 17 - DNA, 24 bp, synthetic construct
  source 1..24: mol_type = other DNA
actccttcctctccgaaaacctcg

SEQ ID NO: 18 - DNA, 26 bp, synthetic construct
  source 1..26: mol_type = other DNA
tgcggaagcgcgagttttattttcga

SEQ ID NO: 19 - DNA, 27 bp, synthetic construct
  source 1..27: mol_type = other DNA
aatttcggacggattttcgtacggttc

SEQ ID NO: 20 - DNA, 23 bp, synthetic construct
  source 1..23: mol_type = other DNA
ttcccgaatccccttcgcctacg

SEQ ID NO: 21 - DNA, 26 bp, synthetic construct
  source 1..26: mol_type = other DNA
ttcgagttcggagacgtaggcgaagg

(19) United States
(12) Patent Application Publication
(10) Pub. No.: US 2025/0179583 A1
(43) Pub. Date: Jun. 5, 2025

(54) METHYLATED DNA MARKERS AND ASSAYS THEREOF FOR USE IN DETECTING COLORECTAL CANCER

(71) Applicants: Cedars-Sinai Medical Center, Los Angeles, CA (US); The Board of Trustees of the Leland Stanford Junior University, Stanford, CA (US)

(72) Inventors: Megan P. Hitchins, Los Angeles, CA (US); Robert W. Haile, Los Angeles, CA (US); Jun Gong, Sherman Oaks, CA (US); Andrew Hendifar, Santa Monica, CA (US); Michael Snyder, Stanford, CA (US); Hayan Lee, Stanford, CA (US)

(73) Assignees: Cedars-Sinai Medical Center, Los Angeles, CA (US); The Board of Trustees of the Leland Stanford Junior University, Stanford, CA (US)

(21) Appl. No.: 18/842,594

(22) PCT Filed: Mar. 2, 2023
(86) PCT No.: PCT/US2023/063594

§ 371 Date: Aug. 29, 2024

Related U.S. Application Data
(60) Provisional application No. 63/315,895, filed on Mar. 2, 2022.

(51) Int. Cl. C12Q 1/6886; C12Q 1/6806; C12Q 1/6809; C12Q 1/6827; C12Q 1/686; C12Q 1/6869

(52) U.S. Cl. CPC C12Q 1/6886; C12Q 1/6806; C12Q 1/6809; C12Q 1/6827; C12Q 1/686; C12Q 1/6869; C12Q 2600/106; C12Q 2600/154

(57) ABSTRACT

Differential methylation profiles of a panel of genes are identified from colorectal carcinoma tissues of colorectal cancer patients compared to normal colon mucosal tissue or to white blood cells from healthy subjects. A high methylation level of cytosines at CpG dinucleotide sites (in CpG-rich regions) of the panel of genetic loci are further validated in plasma cell-free DNA as signature markers indicative of colorectal cancer, which is useful for early stage detection of colorectal cancer and monitoring of disease progression. This panel of genetic loci include genes MAP3K14-AS1, DNM1P46, EMBP1, GATM, VSV2, LAYN, and SFMBT2, which individually or in combination can complement SEPT9-based assays in improving sensitivity and/or specificity in the detection of colorectal cancer. Assay methods and reagents for detection of the presence and levels of methylation of the marker genetic loci are also provided.

### CROSS-REFERENCE TO RELATED APPLICATIONS

This application includes a claim of priority under 35 U.S.C. § 119(e) to U.S. provisional patent application No. 63/315,895, filed Mar. 2, 2022, the entirety of which is hereby incorporated by reference.

### REFERENCE TO SEQUENCE LISTING

This application contains a Sequence Listing submitted as an electronic file named “065472_000891WOPT_SequenceListing.xml”, having a size in bytes of 19,453 bytes, and created on Mar. 1, 2023 (WIPO production date noted as 2023-03-02). The information contained in this electronic file is hereby incorporated by reference in its entirety.

### FIELD OF INVENTION

This invention relates to detection biomarkers for colon and rectal adenocarcinoma and assays thereof including liquid biopsy-based ones for detection in circulating tumor DNAs.

### BACKGROUND

Colonoscopy is considered as a standard test to screen for colorectal cancer (CRC), which has the advantage of being cancer-preventive by allowing for the removal of precancerous lesions (polyps/adenomas). However, colonoscopy is invasive, expensive, and it is not always the first choice for screening purposes. Unfortunately, screening rates among all screen-eligible populations is suboptimal in the U.S. In the most recent study among the general population, screening uptake was 65.1% for any test, 61.7% for colonoscopy, and just 10.4% for a fecal-based test. Reasons for refusing a colonoscopy included concerns about the preparation, adverse risks, embarrassment, and time away. For fecal-based tests, the main aversion was the unpleasant specimen collection. The American Cancer Society (ACS) and most recently (Oct. 28, 2020) the U.S. Preventive Services Task Force (USPSTF) have recommended that population-based screening should begin at 45 years of age, such that average-risk adults aged 45-75 years are now eligible for population-based screening. Clearly, given the high cost and associated risks of colonoscopy, especially among younger persons (<50 years), there is high need to consider more tailored, cost-effective, and non- or minimally-invasive approaches to screening to complement colonoscopy and fecal screening tests, and to identify those for whom a (follow-up) colonoscopy is warranted.

The detection of circulating tumor DNA (ctDNA) shed by cancer cells into the bloodstream provides a way for the detection of cancer-specific signals using blood-based tests. In cancer patients, ctDNA derived from the tumor is detectable by assaying the cell-free DNA (cfDNA) found in blood plasma i.e. cfDNA is found in healthy subjects too, and is derived primarily from dead white blood cells (85%), vascular endothelial cells (10%), liver (2%), and other normal cells (3%). In cancer patients, the ctDNA joins the cfDNA pool and is detectable using cancer-specific biomarkers. Cancer-specific methylated DNA or tumor “somatic” sequence mutations provide tumor-specific biomarkers that will differentiate ctDNA from normal cfDNA, and thereby detect cancer signals.

Existing tests for the detection of colorectal cancer do not offer sufficient accuracy or capabilities for early-stage detection. Epi-proColon (ColoVantage) is a blood-based CRC screening test, used in average-risk adults aged 50-75 years old for the detection of methylated (m) SEPTIN9 gene (also called SEPT9 gene) as ctDNA in plasma. Development of the mSEPT9® test was based on the finding of methylation at high prevalence in CRC, and low or absent methylation in normal colorectal mucosa (NCM) tissue and blood leukocytes, using a low-resolution array-based method. However, the diagnostic performance characteristics of the mSEPT9® test, about a 72% sensitivity and 80% specificity, may fall short of the ≥74% sensitivity and ≥90% specificity diagnostic performance criteria required by the Center for Medicare and Medicaid Services for blood-based biomarker assays (when compared with colonoscopy as the “gold standard” screening test).

The COLVERA test is used in detection of minimal residual disease (MRD) or recurrence based on a two-gene assay of methylated BCAT1 and IKZF1 in colorectal cancer patients following surgical resection or other curative-intent treatment, but is considered suboptimal for population-based screening due to suboptimal diagnostic performance and biases/skewed towards distal located tumors. A multi-cancer detection, “Galleri” test, has low sensitivity at early tumor stages. Current next-generation sequencing (NGS)-based tests are generally expensive, require a long turn-around time, and often cannot be done in-house. For example, NGS-based tests may require prior tumor-based exome sequencing to identify mutations in order for subsequent tracking in plasma, and/or the blood samples to be sent to outside vendors for testing.

Therefore, it is an objective of the present invention to provide a multi-marker panel of biomarkers frequently methylated in tumors, and not in healthy tissues, to increase confidence and improve diagnostic performance in detection test result for colorectal cancer patients.

It is a further objective of the present invention to provide assay kits and methods for detection of colorectal cancer, especially early-stage colorectal cancer, which are relatively inexpensive, easily interpretable, and have a rapid turnaround time.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### SUMMARY OF THE INVENTION

The following embodiments and aspects thereof are described and illustrated in conjunction with compositions and methods which are meant to be exemplary and illustrative, not limiting in scope.

Various embodiments provide methods for methylation analysis of one or more marker genetic loci in a subject in need thereof, or diagnosing colorectal cancer in a subject based on methylation levels of the one or more marker genetic loci, or monitoring progression (or regression) of colorectal cancer in a subject based on methylation levels of the one or more marker genetic loci. The methods generally include measuring a methylation level of one or more of marker genetic loci in a biological sample obtained from the subject, said marker genetic loci comprising one, two, three, four, five, six, or all of genes: DNM1P46, EMBP1, GATM, VS2, MAP3K14-AS1, LAYN, and SFMBT2. In some embodiments, the methods include measuring the methylation level of SEPT9 in addition to the one, two, three, four, five, six, or seven of the above-mentioned genes.

In various embodiments, higher methylation levels are detected of the one or more marker genetic loci relative to respective reference methylation levels. This would indicate diagnosis of colorectal cancer in the subject; and in further embodiments, it would indicate obtaining treatment or intensification of treatment against the colorectal cancer in the subject. Respective reference methylation level can be one measured in normal colon mucosa (NCM) of the subject; or one measured in a control subject free of colorectal cancer or another cancer.

Thus, various embodiments provide methods for treating a subject with colorectal cancer, and the methods include providing a treatment to a subject measured in a biological sample of the subject measured with a methylation level of one or more marker genetic loci above a reference methylation level, wherein the marker genetic loci comprise any one, two, three, four, five, six, or all of genes: DNM1P46, EMBP1, GATM, VSX2, MAP3K14-AS1, LAYN, and SFMBT2; and optionally the subject is also measured with a methylation level of SEPT9 in the biological sample above respective reference methylation level.

Embodiments of methods for treating a subject with colorectal cancer are also provided, and the methods include measuring or requesting measurement of a methylation level of one or more marker genetic loci in a biological sample of the subject, wherein the marker genetic loci comprise any one, two, three, four, five, six, or all of genes: DNM1P46, EMBP1, GATM, VSX2, MAP3K14-AS1, LAYN, and SFMBT2, and optionally further measuring or requesting measuring of a methylation level of SEPT9; and providing a treatment to the subject if the methylation level of the one or more marker genetic loci in the biological sample is above respective reference methylation level.

Additional embodiments provide methods for assaying a subject having undergone surgery or a treatment against colorectal cancer, identifying presence or absence of minimal residual disease in the subject, and/or identifying risk of cancer recurrence or relapse in the subject, and the methods include measuring a methylation level of the one or more marker genetic loci in a biological sample obtained from the subject, said marker genetic loci comprising genes. DNM1P46, EMBP1, GATM, VSX2, MAP3K14-AS1, LAYN, and SFMBT2, optionally the one or more marker genetic loci further comprising SEPT9. Higher methylation levels of the one or more measured maker genetic loci relative to respective reference methylation levels indicate presence of the minimal residual disease and/or risk of cancer recurrence or relapse in the subject.

In exemplary methods for diagnosing colon cancer or rectal cancer in a subject, the methods include measuring a methylation level of one or more marker genetic loci in a biological sample obtained from the subject, said marker genetic loci comprising any one, or two or more, or three or more, or four or more, or five or more, or six or more, or all of genes: DNM1P46, EMBP1, GATM, VSX2, MAP3K14-AS1, LAYN, and SPMBT2; and diagnosing that the subject has a colon cancer or rectal cancer when the methylation level of the one or more marker genetic loci is above respective reference methylation level.

In another exemplary method for diagnosing colon cancer or rectal cancer in a subject, the method includes measuring a methylation level of each marker genetic loci, in a biological sample obtained from the subject, above respective reference methylation levels, thereby diagnosing colon cancer or rectal cancer in the subject, wherein the marker genetic loci comprise genes: DNM1P46, EMBP1, GATM, VSX2, MAP3K14-AS1, LAYN, SPMBT2, or a combination thereof. In some implementations, the method further includes measuring a methylation level of SEPT9 in the biological sample above its reference methylation level.

Embodiments of methods of screening for colon cancer are also provided, which include performing colonoscopy on a subject measured, in a biological sample from the subject, with a methylation level of one or more marker genetic loci above respective reference methylation levels, wherein the one or more marker genetic loci comprise gene: DNM1P46. EMBP1, GATM, VSX2, MAP3K14-AS1, LAYN, SFMBT2, or a combination thereof, and optionally the one or more marker genetic loci further comprising SEPT9.

In various embodiments, a marker genetic locus for methylation level measurement of the invention include two or more, or 10-50, 50-100, 100-200, 200-300, 300-400, 400-500, or more CpG sites in a gene.

Some embodiments provide the marker genetic loci for methylation level measurement have genomic coordinates, within which all of, or at least 95%, 90%, 80%, 70%, 60%, or at least 50% of, the CpG dinucleotides are measured for methylation status:
chr1:121519493-121519559 for EMBP1,
chr15:99806584-99806722 for DNM1P46,
chr15:45378270-45378365 for GATM,
chr14:45378270-74240641 for VSX2,
chr11:111540870-111541174 for LAYN,
chr17:45262243-45262339 for MAP3K14-AS1, and
chr10:7410510-7410579 for SFMBT2.

Additional embodiments provide the marker genetic loci for methylation level measurement are in an extended CpG island region having genomic coordinates:
EMBP1 CpG island range: Chr1:121519060-121519727 (668 bp),
DNM1P46 CpG island range: Chr15:99806520-99807249 (730 bp),
GATM CpG island range: Chr15:4537777345378831 (1059 bp),
VSX2 CpG island range: Chr14:74239486-74241489 (2004 bp),
LAYN CpG island range Chr11:111540208-111541474 (1267 bp),
MAP3K14-AS1 CpG island range: Chr17:45261748-45262475 (728 bp), and
SFMBT2 CpG island range: Chr10:7407415-7413250 (5.836 bp).

In some embodiments, all of the CpG dinucleotides are measured for methylation status within a region defined by the genomic coordinates. Typically bisulfite conversion and qPCR sequencing, or Methyl Light, can be used to measure/detect methylation percentage in a defined genetic loci.

The biological sample in one or more methods disclosed herein contains cell-free DNA (cfDNA). In some instances, it contains circulating tumor DNA (ctDNA).

In some embodiments, the biological sample comprises colorectal mucosa or is obtained from the colorectal mucosa of the subject. In some embodiments, the biological sample comprises plasma or blood, or is obtained from the subject's plasma or blood. In some embodiments, the biological sample is obtained from the subject's feces. In some embodiments, the biological sample comprises tumor tissue or is a biopsy obtained from a cancerous tissue of the subject.

The subject in one or more methods disclosed herein, in various implementations, is a human subject. The subject may be one desiring a determination of colorectal health. The subject in other embodiments is one with a stage I or II colon cancer or stage I or II rectal cancer. The subject in other embodiments is one with a stage III or IV colon cancer or stage III or IV rectal cancer. The subject in other embodiments is one with metastatic colorectal cancer.

Additional embodiments provide methods for assessing efficacy or effectiveness of a treatment to a subject with colorectal cancer, or monitoring progression of the colorectal cancer in the subject, and the methods include measuring a methylation level of one or more marker genetic loci in a first biological sample obtained from the subject at a time t0, measuring a methylation level of the one or more marker genetic loci in a second biological sample obtained from the subject at a time t1, said time t1 being subsequent to said time t0, and for assessing the efficacy or effectiveness of the treatment said time t1 being subsequent to the treatment. The one or more marker genetic loci can be in genes: DNM1P46, EMBP1, GATM, VS2, MAP3K14-AS1, LAYN, or SFMBT2, or a combination thereof, and optionally further in SEPT9. A treatment is indicated to be effective, or the colorectal cancer is indicated to show regression or has not worsened, when DNM1P46, EMBP1, GATM, VSX2, MAP3K14-AS1, LAYN, SFMBT2, and SEPT9, when selected, each has a lower methylation level in the second biological sample obtained at the time t1 relative to that in the first biological sample obtained at the time t0. The colorectal cancer can be indicated to show progression or worsening, when DNM1P46, EMBP1, GATM VSX2, MAP3K14-AS1, LAYN, SFMBT2, and SEPT9, when selected, has a higher methylation level in the second biological sample obtained at the time t1 relative to that in the first biological sample obtained at the time t0.

Measuring methylation level in a genetic locus may be performed by:
treating DNA in the biological sample with one or more reagents to convert unmethylated cytosine bases to uracil sulfonate or another base having a different binding behavior than cytosine, while methylated cytosine bases remain unchanged;
amplifying the treated DNA in the presence of a forward primer oligonucleotide and a reverse primer oligonucleotide, and optionally a polymerase, wherein each of the forward primer oligonucleotide and the reverse primer oligonucleotide hybridizes specifically onto the treated DNA of the one or more marker genetic loci; and
sequencing the amplified DNA in the above step to deduce percentage of methylated cytosine bases in the one or more marker genetic loci as the methylation level.

Additional embodiments provide kits or combinations. The kit or combination may include: a first oligonucleotide which hybridizes onto a first region of one or more marker genetic loci selected from genes of DNM1P46, EMBP1, GATM, VSX2, MAP3K14-AS1, LAYN, SFMBT2, a combination of DNM1P46, EMBP1, GATM, VSX2, MAP3K14-AS1, LAYN, and SFMBT2, and a combination of SEPT9 and one or more of DNM1P46, EMBP1, GATM, VSX2, MAP3K14-AS1, LAYN, and SPMBT2; and a second oligonucleotide which hybridizes onto a second region of each of the selected one or more marker genetic loci; and optionally a polymerase. In some implementations, the first and the second oligonucleotides are each 22-30 bases in length, and each of the first region and the second region comprises at least one CpG dinucleotide site. In some implementations, the first and the second oligonucleotides are selected from a forward PCR primer sequence and a corresponding reverse PCR primer sequence of Table 2. In additional implementations, the kit also includes a third oligonucleotide which hybridizes onto a third region of each of the one or more marker genetic loci. And the third oligonucleotide may be modified with one or more detectable labels, one or more quenchers, or both. In some implementations, the third oligonucleotide is selected from a Probe sequence of Table 2; and wherein the third region includes at least three CpG dinucleotide sites.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts an overview of step-wise discovery and validation of CRC-specific differentially-methylated regions (DMRs) in tissues and plasma to derive new methylated ctDNA markers suitable for the blood-based detection of CRC.

FIG. 2 depicts a Venn diagram showing the intersect of early-stage (stages I and II) tumor and paired normal colorectal mucosa from CRC patients in the discovery series from which high-quality Methyl-Seq data were obtained.

FIG. 3 depicts that principal components analyses (PCA) showed distinct clustering of the methylation profiles of samples from FIG. 2 and white blood cells into three groups by tissue type. Tumor (T) is in gray, distant normal colorectal mucosa (N) in green, and white blood cells (B) in red.

FIG. 4A depicts a sample matrix for colorectal cancer tumor and distant normal colorectal mucosa (normal) samples for which Methyl-Seq data were obtained. Matrix of orthogonal features in colorectal cancer stage I/II tumors showing diverse representation of tumor location, molecular subtypes, age and sex distribution.

FIG. 4B-4L are illustrative validation data for a subset of the biomarkers that are identified as top-ranked differentially methylated regions (DMRs) in our study, as well as the corresponding CpG probe in the 450k array data from the COADREAD dataset from The Cancer Genome Atlas (TCGA), which are closest to (≤250 bp) and within the same CpG-rich feature as the top-ranked DMR(s). These individual CpG probes had highly significant differences in the levels of methylation in primary CRC tissues versus paired normal tissues (but not always recurrent or metastatic tissues) in the COADREAD dataset.

FIG. 4B. SEPTIN9 (which encodes a protein called septin-9) and cg20275528.

FIGS. 4C and 4D: For GATM (which encodes a protein called glycine amidotransferase) the target region contains two CG sites represented on the 450k array, namely cg11431346 & cg01145430.

FIGS. 4E and 4F: SFMBT2 (which encodes Scm Like With Four Mbt Domains 2) and cg01056653 & cg26878816, wherein human methylation 450k array cg sites cg01056653 & cg26878816 flank the target region (of SFMBT2) but lie within the same CpG island.

FIGS. 4G and 4H: MAP3K14-AS1 (SPATA) (MAP3K14 antisense RNA 1) and the closest flanking markers. cg26742995 & cg26532627, cg26742995 & cg26532627 are closest flanking markers to MAP3K14-AS1 (SPATA).

FIG. 4I: EMBP1 (which encodes embigin pseudogene 1) and the closest cg07794500.

FIG. 4J: LAYN (which encodes layilin) and the closest cg03864000.

FIG. 4K: EVL (which encodes Enah/Vasp-Like) and the closest cg23295454.

FIG. 4L: VSX2 (which encodes Visual System Homeobox 2) and the closest cg02084669.

FIG. 4M is a heatmap showing the correlation of methylation levels of individual biomarkers with each of the other biomarkers, all identified in our study of primary tumor tissues, as a way for assessing complementarity to mSEPT9 as the biomarker. For example, MAP3K14-AS1 and SEPTIN9 show low-correlation, meaning they are likely to be complementary (MAP3K14-AS1 is positive in a proportion of tumors that SEPTIN9 is negative for, and vice-versa). Similarly, GATM, DNM1P46, and VSX2 show low correlation with SEPTIN9. Therefore, MAP3K14-AS1, GATM and VSX2, individually or in combination, may complement SEPTIN9. See also Table 6.

FIG. 5 depicts the plasma-based validation of methylated ctDNA markers LAYN (top) and DNM1P46 (Putative GED domain-containing protein DNM1 Pseudogene 46) (bottom). Amplification plots are shown for the methylation-specific real-time PCR assays for LAYN and DNM1P46 (blue traces) in a duplex reaction with cfDNA input control ACTB (purple traces) in plasma from a metastatic CRC case (left), and in plasma from a healthy control (right). The LAYN and DNM1P46 signals are high in plasma from mCRC cases, but are undetectable (flat line) in plasma from healthy controls, indicating they are highly specific for ctDNA detection.

FIG. 6 depicts a case-control study design and recruitment plan to assess the sensitivity and specificity of the plasma-based mSEPT9 and new methylated circulating tumor DNA (mctDNA) markers to detect CRC. (TP, true positive, FP, false positive, TN, true negative, FN, false negative. PCL, precancerous lesion(s).)

FIG. 7A, top, depicts results from a machine learning model using logistic regression of the discovery dataset of colorectal cancer stage I/II tumors versus normal colorectal mucosa (NCM) samples with six methylation markers in six genes: MAP3K14-AS1 (other gene name SPATA32), LAYN, EMBP1, VSX2, GATM, DNM1P46. The logistic regression model has been trained with 80% of samples with 5-fold cross-validation. The accuracy was 0.96, predicted on the rest 20% of data that had never been used either for training or cross-validation. The receiver operating characteristic curve (ROC) shows 0.83 (test) and 0.96 (validation) of the area under the ROC curve (AUC). FIG. 7A, bottom, depicts weights of methylation biomarker features visualized as a histogram. The methylation biomarker locus MAP3K14-AS1 shows the most significant weight, followed by LAYN, EMBP1, VSX2, and GATM. The prediction outcome may slightly alter based on size of sample sets.

FIG. 7B, top, depicts results from a machine learning model using logistic regression of the discovery dataset of colorectal cancer stage I/II tumors versus normal colorectal mucosa (NCM) samples with five non-redundant methylation markers in five genes: MAP3K14-AS1 (other gene name SPATA32), EMBP1, VSX2, GATM, DNM1P46. The logistic regression model has been trained with 80% of samples with 5-fold cross-validation. The accuracy was 0.96, predicted on the rest 20% of data that had never been used either for training or cross-validation. The receiver operating characteristic curve (ROC) shows 0.96 (validation) and 0.83 (test) of the area under the ROC curve (AUC). FIG. 7B, bottom, depicts weights of methylation biomarker features visualized as a histogram. The methylation biomarker locus MAP3K14-AS shows the most significant weight, followed by VSX2 then EMBP1 then GATM, DNM1P46 adds least value to the combination. The prediction outcome may slightly alter based on size of sample sets.

FIG. 8, top, depicts results from re-analysis of a machine learning model using logistic regression of the discovery dataset of colorectal cancer stage I/II tumors versus normal colorectal mucosa (NCM) samples with five non-redundant methylation markers in five genes: MAP3K14-AS1 (other gene name SPATA32), EMBP1, VSX2, GATM, and DNM1P46, plus mSEPTIN9. The logistic regression model has been trained with 80% of samples with 5-fold cross-validation. The accuracy was 0.96, predicted on the rest 20% of data that had never been used either for training or cross-validation. Interestingly, the addition of mSEPT9 did not increase the accuracy of this panel of five non-redundant markers. The receiver operating characteristic curve (ROC) remained at 0.96 (test) and 0.83 (training) of the area under the ROC curve (AUC). FIG. 8B, bottom, depicts weights of the same five plus mSEPT9 methylation biomarker features visualized as a histogram, VSX2 shows the most significant weight (added value), followed by MAP3K14-AS1 (other gene name SPATA32), then FMBP. GATM and DNM1P46 are of least added value when SEPTIN9 is included. The prediction outcome may slightly alter based on size of sample sets.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed, Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The CpG sites or CG sites, shorthand for 5′-C-phosphate-G-3′ (cytosine and guanine separated by only one phosphate group), are regions of DNA where a cytosine nucleotide is followed by a guanine nucleotide in the linear sequence of bases along its 5′→3′ direction. This single-stranded linear dinucleotide sequence is distinguished from the CG base-pairing of cytosine and guanine for double-stranded sequences. CpG sites occur with high frequency in genomic regions called CpG islands (or CG islands). Cytosines in CpG dinucleotides can be methylated to form 5-methylcytosines. Enzymes that add a methyl group are called DNA methyltransferases. Methylated cytosines often mutate to thymines. Methylating the cytosine within a gene can change its expression, a mechanism in gene regulation called epigenetics.

CpG islands (or CG islands) are regions with a high frequency of CpG sites. Generally, it is a region with at least 200 bp, a GC percentage greater than 50%, and/or an observed-to-expected CpG ratio greater than 60%. CpG islands as DNA methylated regions in promoters can regulate gene expression through transcriptional silencing of the corresponding gene. The presence of multiple methylated CpG sites in CpG islands of promoters can cause stable silencing of genes. In cancers, loss of expression of genes occurs much more frequently by hypermethylation of promoter CpG islands than by mutations. Conversely, hypomethylation of CpG islands in promoters results in overexpression of the genes or gene sets affected.

Genes, marker genes, or gene markers, may be polynucleotides that are genomic DNA, cDNA, or mRNA transcripts, and in a broad sense, include pseudogenes. An alternative term is “genetic loci,” which include functional genes and pseudogenes. The polynucleotide may contain deoxyribonucleotides, ribonucleotides, and/or their analogs and may be double-stranded or single stranded. A polynucleotide can comprise modified nucleic acids (e.g., methylated), nucleic acid analogs or non-naturally occurring nucleic acids and can be interrupted by nonnucleic acid residues. For example, a polynucleotide includes a gene, a gene fragment, cDNA, isolated DNA, mRNA, tRNA, rRNA, isolated RNA of any sequence, recombinant polynucleotides, primers, probes, plasmids, and vectors. Unless otherwise noted, genes expressed by gene symbols (e.g., italicized with all letters in uppercase for human gene symbols) include functional genes and pseudogenes.

Generally, pseudogene may refer to a DNA sequence that resembles a functional gene but has been mutated into an inactive form (over the course of evolution). It often lacks introns and other essential DNA sequences necessary for function. Typically, pseudogenes do not result in functional proteins, although some may have regulatory effects.

One way to designate genetic loci. e.g., for those measured for methylation presence or level as disclosed herein, is to use an hg38 coordinate, which is an ID used for Genome Reference Consortium Human Reference 38 (GRCh Build 38) such as in the context of the UCSC Genome Browser. GRCh Build 38 is the primary genome assembly in GenBank. For example, methylation levels of genetic loci, EMBP1, DNM1P46, GATM, VSX2, LAYN, MAP3K14-AS1, and SPMBT2, are measured at or near the hg38 coordinates listed in Table 2.

Another way to designate CpG loci is Illumina's method based on the actual or contextual sequence of each individual CpG locus, described in www.illumina.com/content/dam/illumina-marketing/documents/productsitechnotes/technote_cpg_loci_identification.pdf. It takes advantage of sequences flanking a CpG locus to generate a unique CpG locus cluster ID (cg #), which is unaffected by genome version. Flanking sequences of 60 bases on each side of the CpG locus constitute a 122-base sequence used to define the locus. Any ambiguous nucleotide bases (e.g., N) in this flanking sequence are included. A unique “CpG cluster number” or cg # is assigned to each unique 122-base CpG locus. A single CpG cluster can have multiple members that map onto different loci in a genome only if they have identical sequences.

Within a CpG cluster, three pieces of information, i.e, chromosome number, genomic coordinate, and genome build, are used to track individual member CpG loci. Unless otherwise stated, the CpG loci and their methylation status disclosed herein refer to those in the human genome build version of hg38. For a genomic coordinate, since a CpG locus contains two nucleotides and there are two genomic coordinates for a given site: one for C and the other for G, the lesser of the two coordinates is used as the coordinate of the CpG locus. In various embodiments, custom methylation reagents or products provided herein are designed to target unique CpG sites, i.e., CpG clusters that have only a single member.

In addition to the cg #identifier assignment, the TOP/BOT strandedness of each CpG locus can be determined, using a similar TOP/BOT strand nomenclature commonly seen in SNP strand designation. For CpG loci, both the C and G of the CpG locus are treated as a single unit, and the CpG dinucleotide is defined as position ‘n’. The bases immediately before and after the CpG are ‘n−1’ and ‘n+1’, respectively. Similarly, the second base before the CpG is ‘n−2’ and the second base after the CpG is ‘n+2’, etc. The designation of TOP or BOT strand for CG sites uses a sequence walking method, in which sequence walking continues until a first unambiguous pairing is present. An unambiguous pair is two bases equidistant from the CpG, one (and only one) of which is an A or T (i.e., A/G, A/C. T/C, or T/G). If the A or T in the first unambiguous pair is on the 5′ side of the CpG, then the sequence is designated TOP. If the A or T in the first unambiguous pair is on the 3′ side of the CpG, then the sequence is designated BOT.

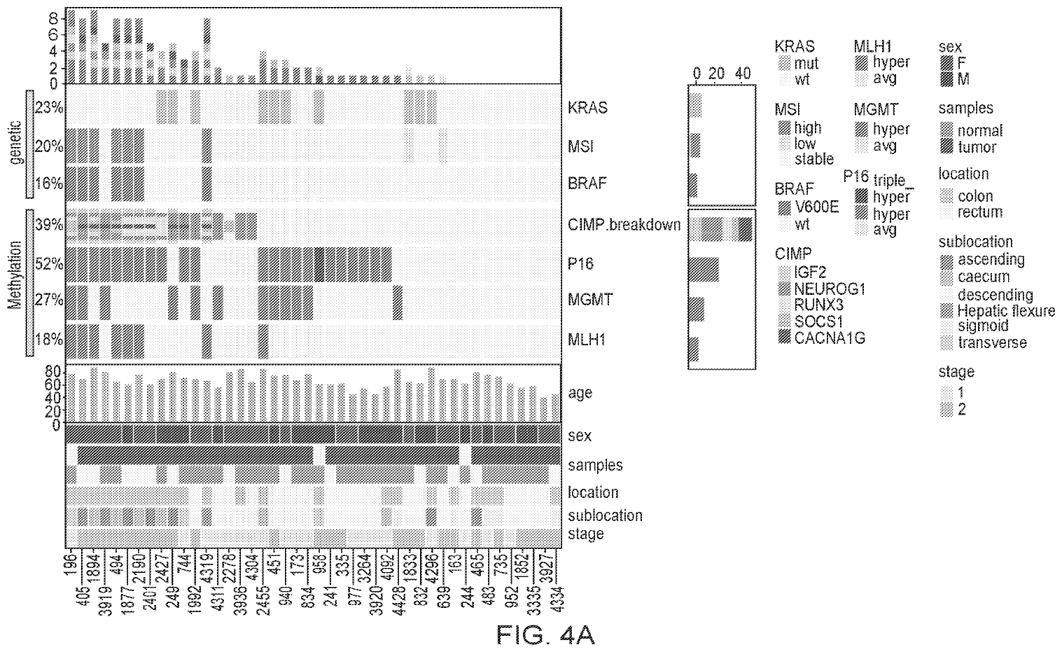

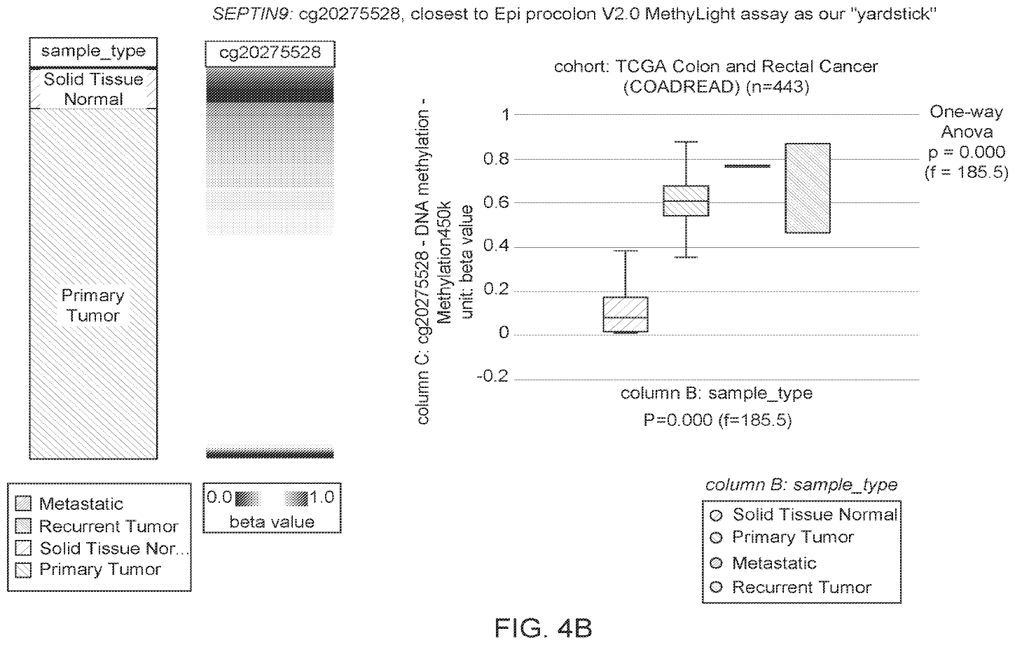

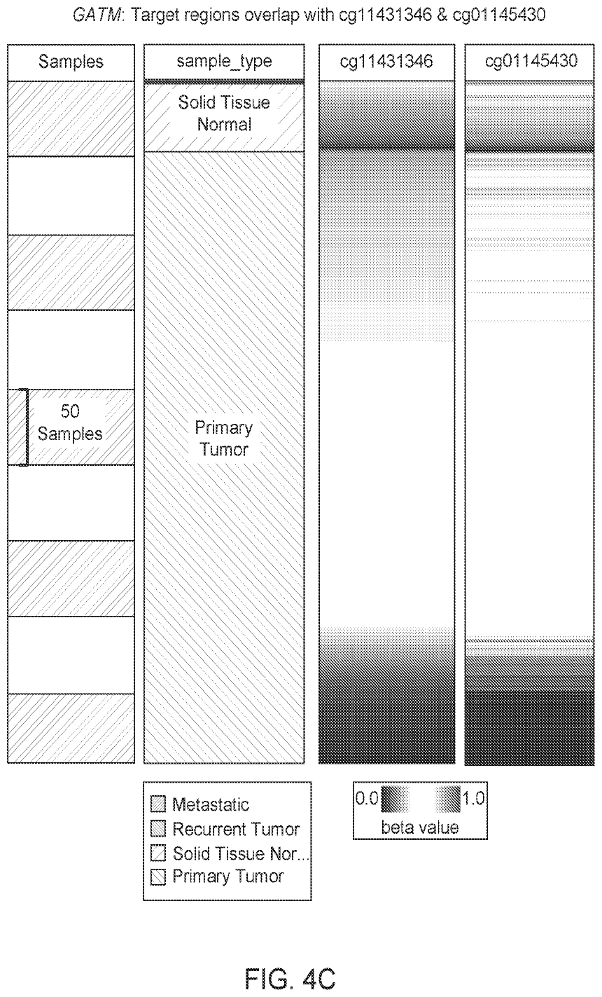

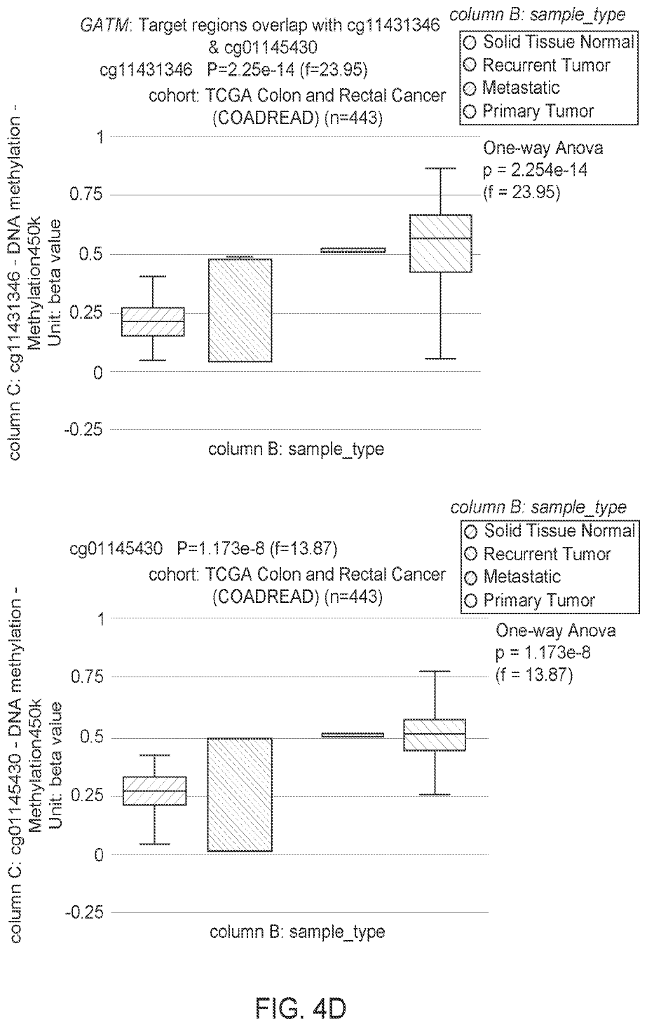

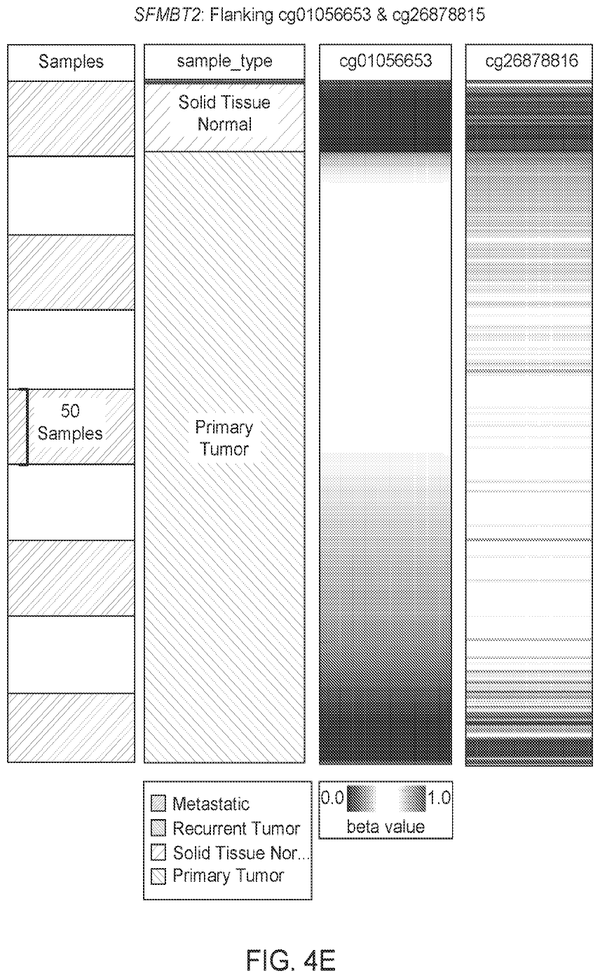

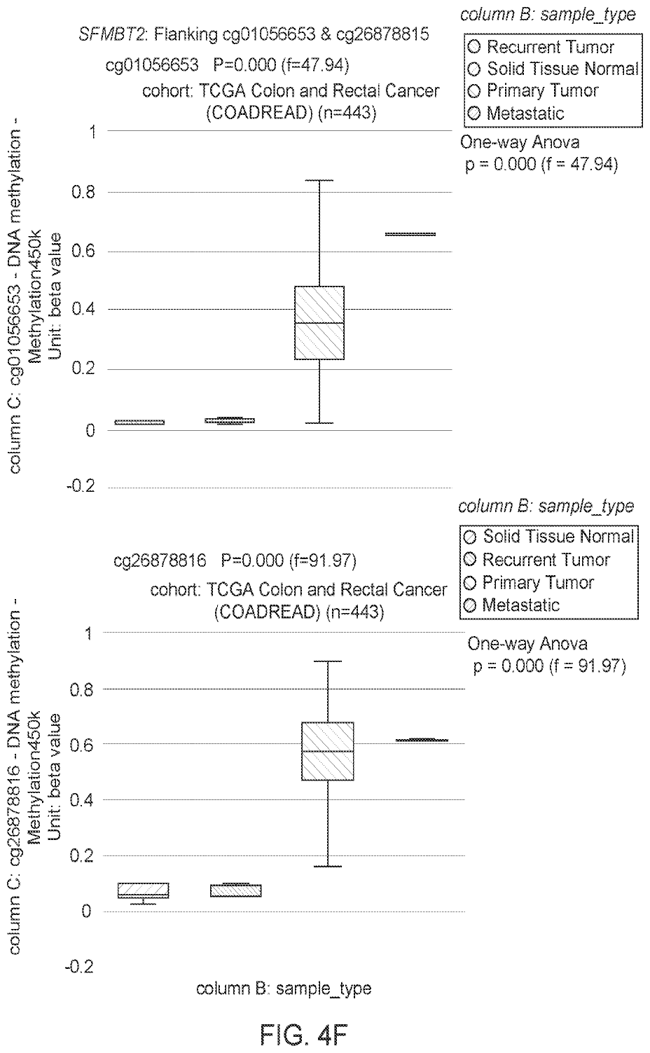

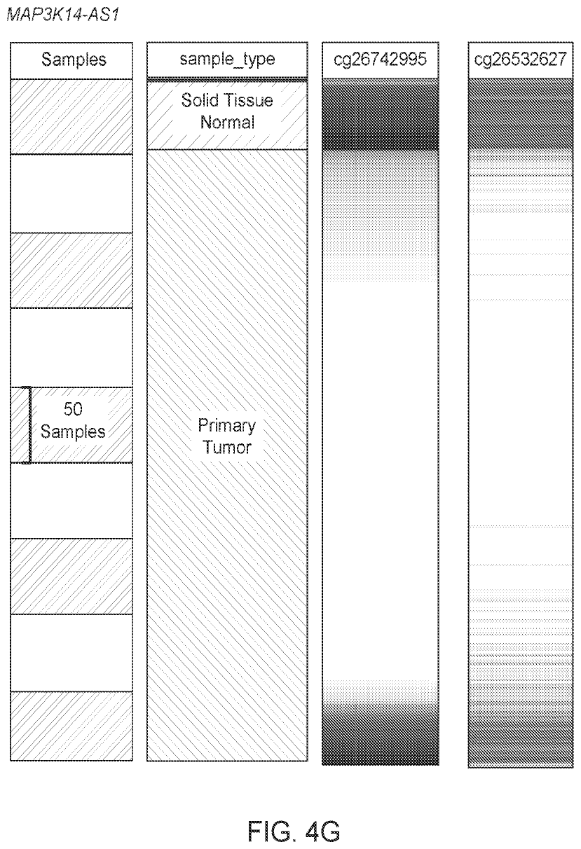

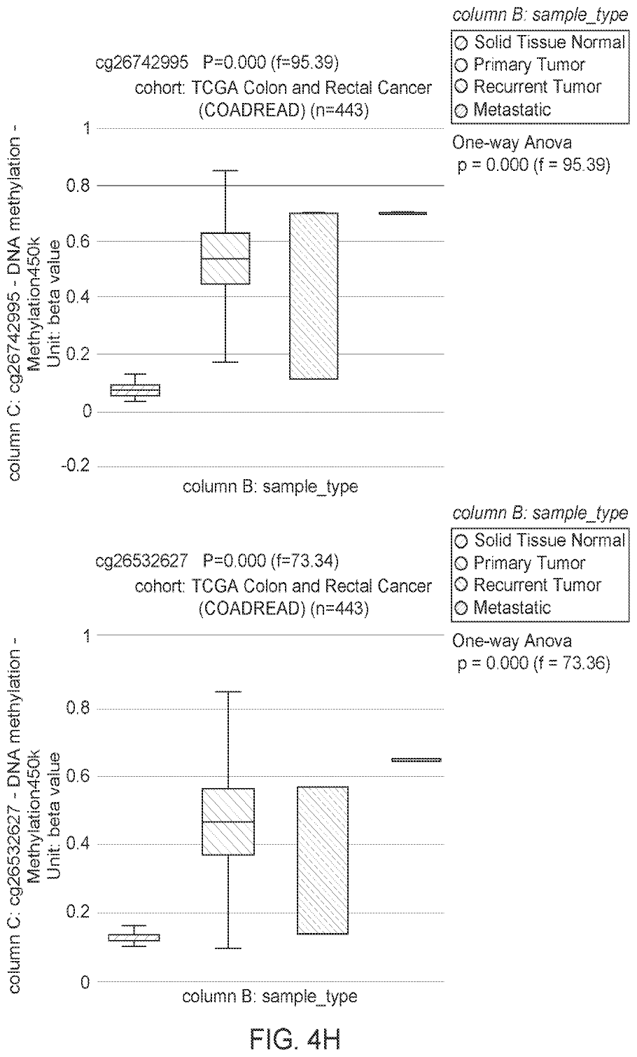

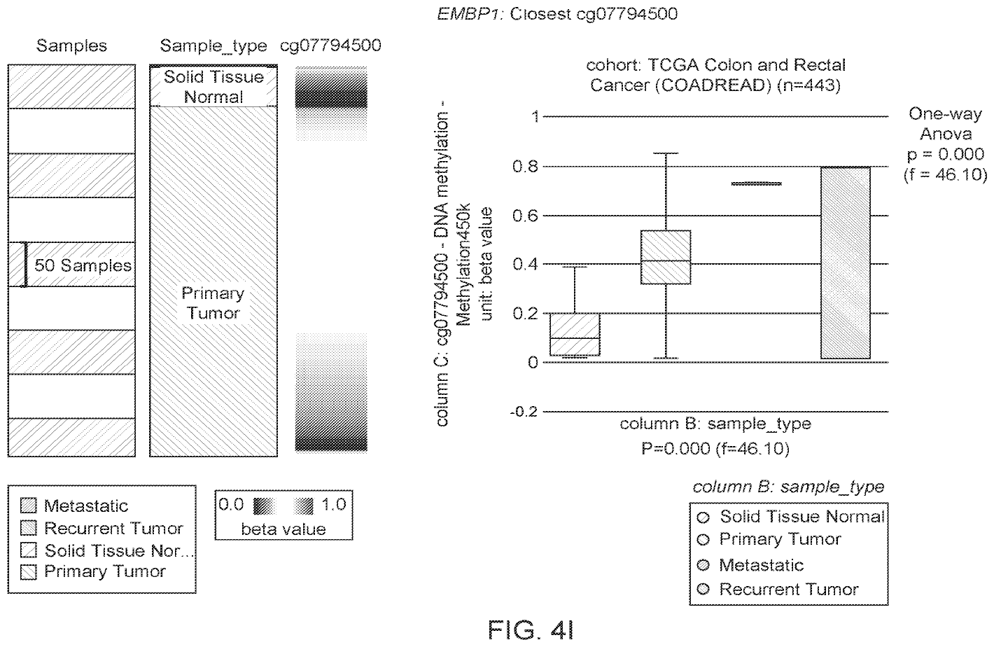

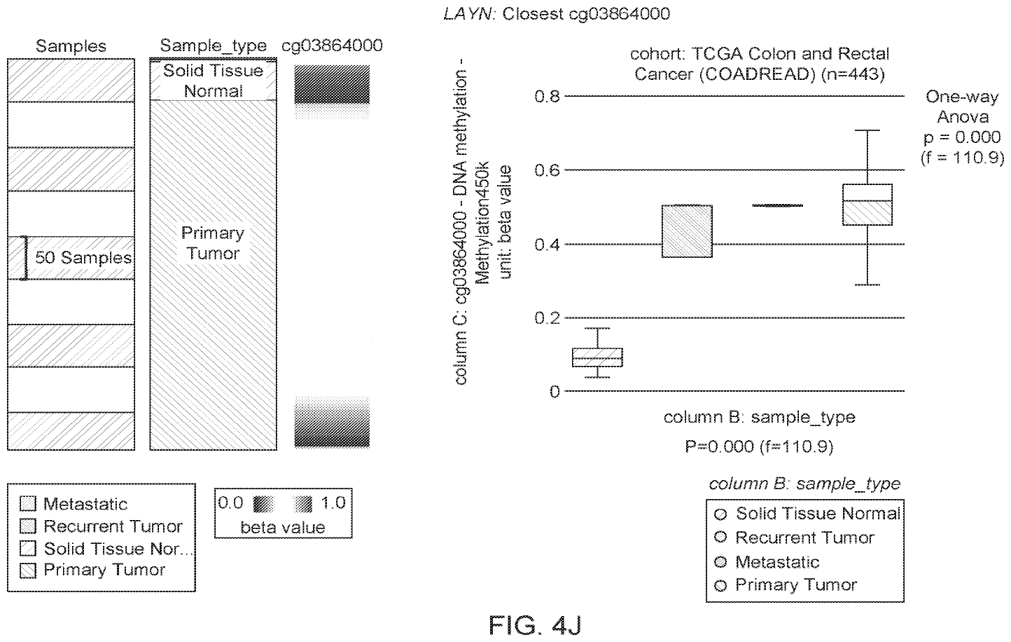

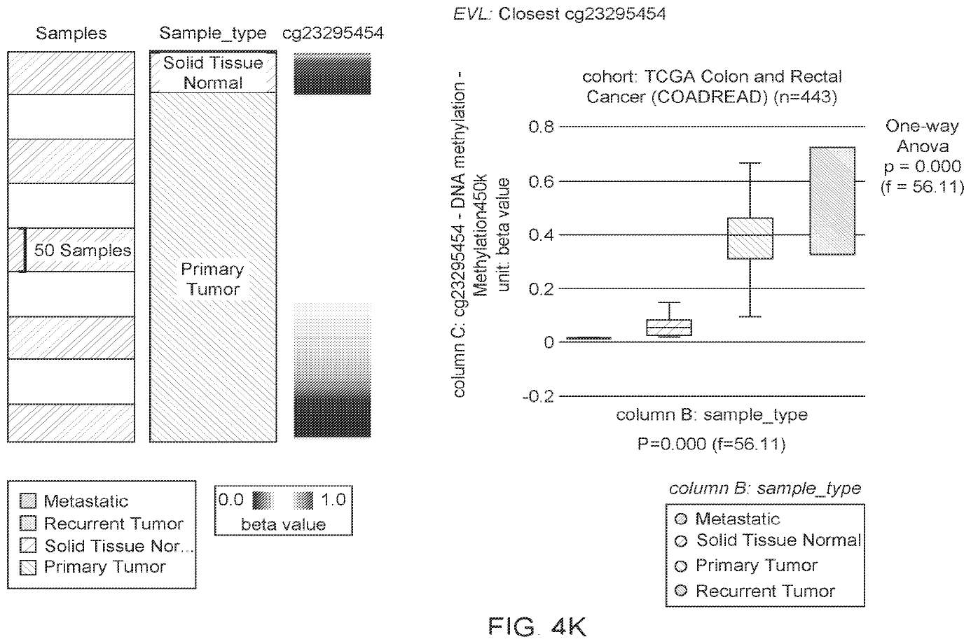

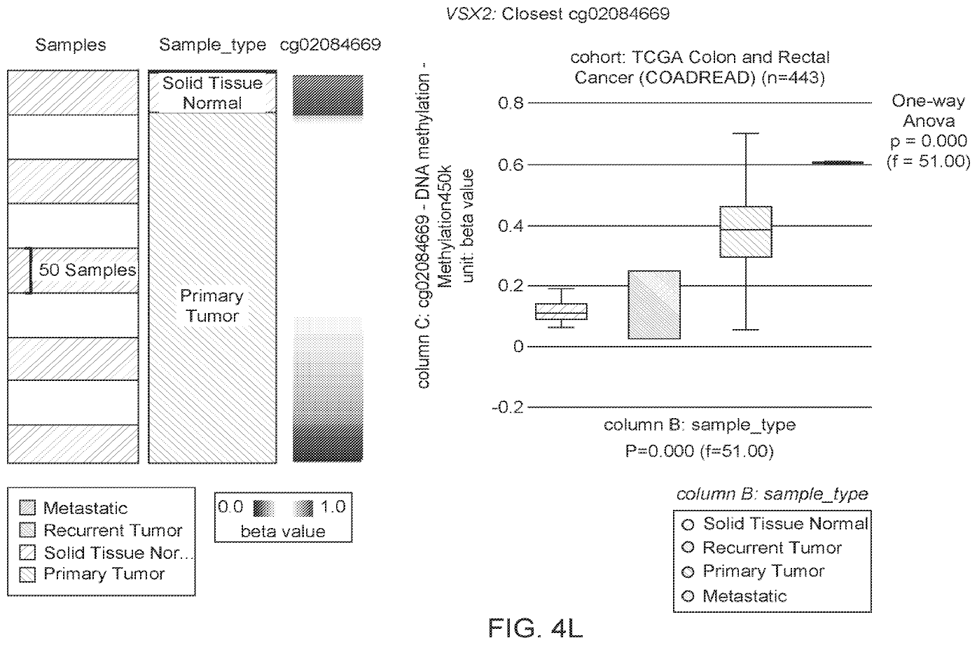

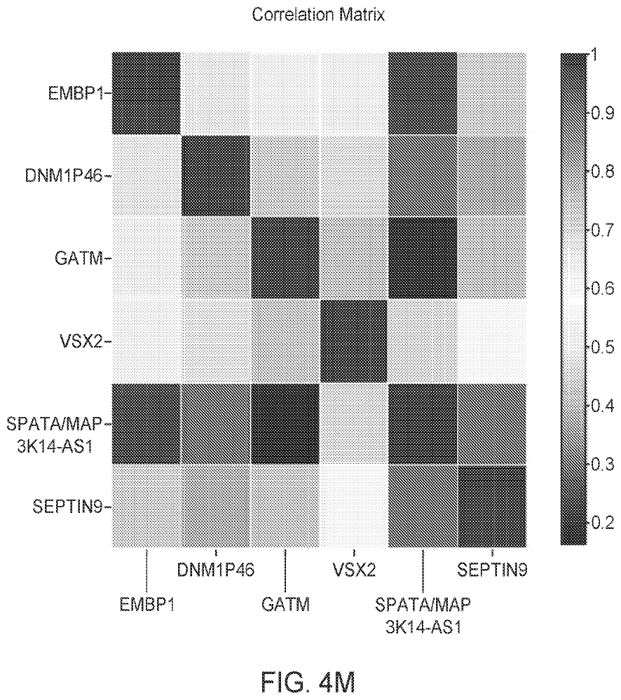

As referred to in figures such as FIG. 4B, beta-value describes the methylation fraction (between 0 and 1, whereby 0 is unmethylated, 1 is completely methylated) for a given locus across a population of cells. DNA methylation occurs in human at CpG dinucleotides where the ‘C’ can be methylated or not. The methylation state of a given locus in a single cell is typically binary (although technically tertiary as there are two copies of most chromosomes). When measured across a population of cells, some loci may have intermediate methylation values (between 0 and 1), and the methylation percentage (or Beta-value) is used to describe this.

Bisulfite genomic sequencing is a technique for detection of DNA methylation. The amination reactions of cytosine and 5-methylcytosine (5 mC) proceed with very different consequences after the treatment of sodium bisulfite. Cytosines in single-stranded DNA will be converted into uracil residues and recognized as thymine in subsequent PCR amplification and sequencing (after PCR amplification, uracil residues are converted to thymine), however, 5 mCs are immune to this conversion and remain as cytosines allowing 5 mCs to be distinguished from unmethylated cytosines. A subsequent PCR process is often necessary to determine the methylation status in the loci of interest by using specific methylation primers after the bisulfite treatment. The actual methylation status can be determined either through direct PCR product sequencing (detection of average methylation status) or sub-cloning sequencing (detection of single molecules distribution of methylation patterns). Alternatively, methylation-specific PCR assays (used in Examples herein) will amplify DNA templates only if they are methylated.

“Circulating tumor DNA” or “ctDNA” is tumor-derived fragmented DNA in the bloodstream that is not associated with cells. “cell-free DNA” or “cfDNA” is a broader term which describes DNA that is freely circulating in the bloodstream or other biological fluids, but is not necessarily of tumor origin.

A biological sample obtained from a subject can be cell lines, histological slides, biopsies, paraffin-embedded tissue, body fluids, urine, blood plasma, blood serum, whole blood, cells isolated from the blood, and any combinations thereof. In some embodiments, a biological sample is blood plasma. In some embodiments, a biological sample is biopsy from colorectal cancer tissue or normal colorectal mucosal tissue. In some embodiments, a biological sample is isolated nucleic acids from feces.

The earliest stage colorectal cancers are called stage 0 (a very early cancer), and then range from stages 1 (1) through IV (4). Although each person's cancer experience is unique, cancers with similar stages tend to have a similar outlook and are often treated in much the same way. A staging system most often used for colorectal cancer is the American Joint Committee on Cancer (AJCC) TNM system, which is based on 3 key pieces of information: (1) The extent (size) of the tumor (T), characterized by how far the cancer has grown into the wall of the colon or rectum. These layers, from the inner to the outer, include: the inner lining (mucosa), which is the layer in which nearly all colorectal cancers start, this includes a thin muscle layer (muscularis mucosa); the fibrous tissue beneath this muscle layer (submucosa); a thick muscle layer (muscularis propria); and The thin, outermost layers of connective tissue (subserosa and serosa) that cover most of the colon but not the rectum. (2) The spread to nearby lymph nodes (N). (3) The spread (metastasis) to distant sites (M), whether or not the cancer has spread to distant lymph nodes or distant organs such as the liver or lungs.

TABLE 1
An AJCC system for staging colorectal cancer effective January 2018. (*The
following additional categories are not listed: TX: Main tumor cannot be
assessed due to lack of information; T0: No evidence of a primary tumor;
and NX: Regional lymph nodes cannot be assessed due to lack of information.)

| AJCC | Stage | |
|---|---|---|
| Stage | grouping | Stage description* |
| 0 | Tis | The cancer is in its earliest stage. This stage is also known as |
| | N0 | carcinoma in situ or intramucosal carcinoma (Tis). It has not |
| | M0 | grown beyond the inner layer (mucosa) of the colon or rectum. |
| I | T1 or T2 | The cancer has grown through the muscularis mucosa into the |
| | N0 | submucosa (T1), and it may also have grown into the muscularis |
| | M0 | propria (T2). It has not spread to nearby lymph nodes (N0) or to |
| | | distant sites (M0). |
| IIA | T3 | The cancer has grown into the outermost layers of the colon or |
| | N0 | rectum but has not gone through them (T3). It has not reached |
| | M0 | nearby organs. It has not spread to nearby lymph nodes (N0) or to |
| | | distant sites (M0). |
| IIB | T4a | The cancer has grown through the wall of the colon or rectum but |
| | N0 | has not grown into other nearby tissues or organs (T4a). It has not |
| | M0 | yet spread to nearby lymph nodes (N0) or to distant sites (M0). |
| IIC | T4b | The cancer has grown through the wall of the colon or rectum |
| | N0 | and is attached to or has grown into other nearby tissues or |
| | M0 | organs (T4b). It has not yet spread to nearby lymph nodes (N0) or |
| | | to distant sites (M0). |
| IIIA | T1 or T2 | The cancer has grown through the mucosa into the submucosa |
| | N1/N1c | (T1), and it may also have grown into the muscularis propria |
| | M0 | (T2). It has spread to 1 to 3 nearby lymph nodes (N1) or into |
| | | areas of fat near the lymph nodes but not the nodes themselves |
| | | (N1c). It has not spread to distant sites (M0). |
| | OR | |
| | T1 | The cancer has grown through the mucosa into the submucosa |
| | N2a | (T1). It has spread to 4 to 6 nearby lymph nodes (N2a). It has not |
| | M0 | spread to distant sites (M0). |
| IIIB | T3 or T4a | The cancer has grown into the outermost layers of the colon or |
| | N1/N1c | rectum (T3) or through the visceral peritoneum (T4a) but has not |
| | M0 | reached nearby organs. It has spread to 1 to 3 nearby lymph |
| | | nodes (N1a or N1b) or into areas of fat near the lymph nodes but |
| | | not the nodes themselves (N1c). It has not spread to distant sites |
| | | (M0). |
| | OR | |
| | T2 or T3 | The cancer has grown into the muscularis propria (T2) or into the |
| | N2a | outermost layers of the colon or rectum (T3). It has spread to 4 to |
| | M0 | 6 nearby lymph nodes (N2a). It has not spread to distant sites (M0). |
| | OR | |
| | T1 or T2 | The cancer has grown through the mucosa into the submucosa |
| | N2b | (T1), and it might also have grown into the muscularis propria |
| | M0 | (T2). It has spread to 7 or more nearby lymph nodes (N2b). It has |
| | | not spread to distant sites (M0). |
| IIIC | T4a | The cancer has grown through the wall of the colon or rectum |
| | N2a | (including the visceral peritoneum) but has not reached nearby |
| | M0 | organs (T4a). It has spread to 4 to 6 nearby lymph nodes (N2a). It |
| | | has not spread to distant sites (M0). |
| | OR | |
| | T3 or T4a | The cancer has grown into the outermost layers of the colon or |
| | N2b | rectum (T3) or through the visceral peritoneum (T4a) but has not |
| | M0 | reached nearby organs. It has spread to 7 or more nearby lymph |
| | | nodes (N2b). It has not spread to distant sites (M0). |
| | OR | |
| | T4b | The cancer has grown through the wall of the colon or rectum |
| | N1 or N2 | and is attached to or has grown into other nearby tissues or |
| | M0 | organs (T4b). It has spread to at least one nearby lymph node or |
| | | into areas of fat near the lymph nodes (N1 or N2). It has not |
| | | spread to distant sites (M0). |
| IVA | Any T | The cancer may or may not have grown through the wall of the |
| | Any N | colon or rectum (Any T). It might or might not have spread to |
| | M1a | nearby lymph nodes. (Any N). It has spread to 1 distant organ |
| | | (such as the liver or lung) or distant set of lymph nodes, but not |
| | | to distant parts of the peritoneum (the lining of the abdominal |
| | | cavity) (M1a). |
| IVB | Any T | The cancer might or might not have grown through the wall of |
| | Any N | the colon or rectum (Any T). It might or might not have spread to |
| | M1b | nearby lymph nodes (Any N). It has spread to more than 1 distant |
| | | organ (such as the liver or lung) or distant set of lymph nodes, |
| | | but not to distant parts of the peritoneum (the lining of the |
| | | abdominal cavity) (M1b). |
| IVC | Any T | The cancer might or might not have grown through the wall of |
| | Any N | the colon or rectum (Any T). It might or might not have spread to |
| | M1c | nearby lymph nodes (Any N). It has spread to distant parts of the |
| | | peritoneum (the lining of the abdominal cavity), and may or may |
| | | not have spread to distant organs or lymph nodes (M1c). |

“Minimal residual disease (MRD)” refers to a small number of cancer cells that remain in body after cancer treatment, but which are undetectable by standard of care radioimaging methods. These remnant cells have the potential to cause cancer “recurrence” or “relapse”.

Existing plasma-based CRC detection tests use markers that were initially discovered using low-resolution bead-array data that encompassed 14,000 to 450,000 CpG sites (depending on the array used). Our discovery interrogated 4.2 million CpG sites for methylation differences, providing a much higher-resolution of genome-wide methylation markers. We have identified, in early-stage tumors (AJCC pathology stages I and II), methylation markers that are present at early stages of tumor development to improve sensitivity for cancer detection at early stages of disease.

We have developed a panel of various methylated DNA biomarkers and assays for the detection of colorectal cancer (CRC) tumor-derived signals, including circulating tumor DNA in plasma for the detection and measurement of colorectal cancer signals. Tumor-derived DNA methylation and genetic mutations provide target analytes for ctDNA detection. Specifically, cancer-specific methylated DNA is a superior source of biomarkers (than tumor mutations) for ctDNA detection in population-based screening for the early detection of cancer, because the former is highly prevalent across patients with given cancer type, occurs early in tumorigenesis, and provides a more stable analyte (more nuclease-resistant) in plasma.

Some of the biomarkers—including one or both of EMBP1 and DNM1P46—are highly prevalent (“universal”) in CRC, such that they are able to detect the presence of the majority of CRC subtypes. These biomarkers show similar CRC detection diagnostic performance as the existing methylated SEPTIN9 (mSEPTIN9) test. Other biomarkers—including one or more of VSX2, GATM, and MAP3K14-AS1—are complementary to SEPTIN9, i.e. predicted to be frequently present in tumors where methylated SEPTIN9 is absent.

We have considered methylated DNA markers of CRC, other than SEPT9, for use in plasma-based ctDNA assays for the blood-based detection of CRC. In our opinion, the approaches previously used for biomarker discovery were not sufficiently rigorous because they: (1) used low-resolution techniques available at that time (e.g. mSEPT9 was discovered using the first-generation methylation array that interrogated just 14k CpG sites genome-vide), whereas the Methyl-Seq platform we have used is high-resolution, providing bisulfite sequencing data across 4.2 million CpG sites: (2) were conducted in non-ideal samples, including CRC cell lines (prone to culture-based artefacts), or small sample sizes of predominantly advanced-stage (III/IV) CRC, or in plasma samples from metastatic CRC cases, thereby selecting markers of advanced-stage disease. For example, the methylated BCAT1 and IKZF/markers in the COLVERA™ test were originally identified using a prevalence threshold of just 50% of CRC. Unsurprisingly, therefore, BCAT1 and IKZF1 demonstrate a preference for the detection of CRC exhibiting the CpG island methylator phenotype (CIMP), a molecular subtype of CRC that is associated with older age. Furthermore, the plasma-based assays for these two markers consistently produce low-level signals in healthy control plasma, thereby complicating interpretation.

Various embodiments provide methylated cfDNA biomarkers for identification of CRC in a subject, or monitoring disease (with or without treatment) in a subject with a prior diagnosis of CRC. In various aspects, the cfDNA biomarkers are ctDNA derived from and/or indicative of colorectal cancer of the subject. The relatively rapid decay of ctDNA in blood permits it to be biomarkers for real time assessment of any changes in tumor burden. In various implementations, a multi-marker panel of high-prevalence methylated DNA biomarkers is provided for early detection of CRC in blood or fecal samples. In some implementations, an early-stage CRC is detected via the multi-marker panel of methylated DNA biomarkers provided herein, and the early-stage CRC is localized stages I and/or II. In some embodiments, the methylation biomarkers or methylation of genetic loci provided herein are for use in detection or diagnosing of stage I CRC with a blood or plasma sample. In some embodiments, the methylation biomarkers or methylation of genetic loci provided herein are for use in detection or diagnosing of stage I CRC with a fecal sample. In some embodiments, the methylation biomarkers or methylation of genetic loci provided herein are for use in detection or diagnosing of stage II CRC with a blood or plasma sample. In some embodiments, the methylation biomarkers or methylation of genetic loci provided herein are for use in detection or diagnosing of stage II CRC with a fecal sample. In various implementations, the biomarkers are sourced from a subject with tumors of early pathological stage (AJCC pathology stage I and stage II), and therefore can be used to test in an asymptomatic subject to determine if the subject has early stage CRC or related carcinoma. Therefore, in some embodiments, the methylation biomarkers or methylation of genetic loci provided herein are for use in detection or diagnosing of stage III CRC with a blood or plasma sample. In some embodiments, the methylation biomarkers or methylation of genetic loci provided herein are for use in detection or diagnosing of stage III CRC with a fecal sample. In some embodiments, the methylation biomarkers or methylation of genetic loci provided herein are for use in detection or diagnosing of stage IV CRC with a blood or plasma sample. In some embodiments, the methylation biomarkers or methylation of genetic loci provided herein are for use in detection or diagnosing of stage IV CRC with a fecal sample. In some embodiments, the methylation biomarkers or methylation of genetic loci provided herein are for use in detection or diagnosing CRC that has metastasized.

In further implementations, a multi-marker panel of high-prevalence methylated DNA biomarkers is used for detection of CRC regardless of the stage/severity of the CRC. In the Examples, we used stage I and stage II samples for a DISCOVERY set (by 4.2 Million CpG site sequencing), and also demonstrated in a Validation set (COAREAD FIG. 4) that these markers were also retained in later stage tumors (stages III and IV-metastases).

Some embodiments provide a method for methylation analysis of one or more genetic loci in a subject in need thereof, and the method includes measuring a methylation level of the one or more (or a panel of) genetic loci in a biological sample obtained from the subject, said one or more genetic loci comprising gene or pseudogene of: DNM1P46, EMBP1, GATM, VSX2, MAP3K4-AS1, LAYN, SFMBT2, or a combination thereof, wherein the subject has colorectal cancer or requests determination of colorectal cancer.

Further embodiments provide that the method for methylation analysis includes measuring a hypermethylation level of the one or more marker genetic loci, wherein the hypermethylation level refers to at least 1%, 5%, 10%, 20%, 30%, 40%, or 50% higher than a reference methylation level in a normal colon mucosal tissue from the same subject or in white blood cells of a healthy control subject free of cancer. In some aspects, the hypermethylation level is at least 30% higher than the reference methylation level. In various implementations, a 50% (or 30%, or 10% etc.) higher (hyper-)methylation level at a genetic locus in a test sample than in a reference sample describes that the methylation percentage for the given genetic locus across the population of cells in the test sample is 50% (or 30%, or 10% etc.) higher than the methylation percentage across the population of cells in the reference sample. In some embodiments, a reference methylation level is 25% methylation, as shown for the assay result presented Table 6. In other embodiments, a reference methylation level is a “cut-off” level of 0.01% methylation level, as shown in the plasma measurement presented in Table 3.

In some embodiments, the panel of marker genetic loci (or biomarkers) include or are one or both of EMBP1 and DNM1P46, and detection of hypermethylation level of one or both of these genes in addition to presence of mSEPTIN9 in a biological sample improves the specificity of colorectal cancer detection compared to a detection based on mSEPTIN9 only. In other embodiments, detection of hypermethylation in both of EMBP1 and DNM1P46 indicates a high likelihood of a colorectal cancer. In further aspects, detection of hypermethylation in both of EMBP1 and DNM1P46, without detecting methylation status of SEPT9, indicates a high likelihood of a colorectal cancer. In other embodiments, detection of hypermethylation of EMBP1 indicates a high likelihood of a colorectal cancer. In further aspects, detection of hypermethylation of EMBP1, without detecting methylation status of SEPT9, indicates a high likelihood of a colorectal cancer. In other embodiments, detection of hypermethylation of DNM1P46 indicates a high likelihood of a colorectal cancer. In further aspects, detection of hypermethylation of DNM1P46, without detecting methylation status of SEPT9, indicates a high likelihood of a colorectal cancer. In some embodiments, a detected hypermethylation of one or both of EMBP1 and DNM1P46 results in a specificity of at least 90%, 92%, 95%, or 98% in the detection of a colorectal cancer.

In some embodiments, the panel of marker genetic loci include or are one, two, or all of VSX2, GATM, and MAP3K14-AS1 are provided, and detection of hypermethylation of one or more of these genes in addition to presence of mSEPTIN9 in a biological sample improves the sensitivity of colorectal cancer detection compared to a detection based on mSEPTIN9 only. In some embodiments, a detected hypermethylation of one, two, or all of VSX2, GATM, and MAP3K14-AS1 results in a sensitivity of at least 74%, 80%, 85%, or 90% in the detection of a colorectal cancer, when combined with hypermethylation of SEPTIN9 (also referred to as SEPT9). In other embodiments, a method includes measuring in a sample the methylation status of both GATM and SEPT9 and detecting hypermethylation in at least one or both of the GATM and the SEPT9, wherein the detected hypermethylation in at least one or both of the GATM and the SEPT9 indicates a high likelihood of a colorectal cancer. In some aspects, detecting a presence of hypermethylation of GATM and an absence of hypermethylation of SEPT9 can still indicate a high likelihood of a colorectal cancer. In other embodiments, a method includes measuring in a sample methylation the status of both VSX2 and SEPT9 and detecting hypermethylation in at least one or both of the VSX2 and the SEPT9, wherein the detected hypermethylation in at least one or both of the VSX2 and the SEPT9 indicates a high likelihood of a colorectal cancer. In some aspects, detecting a presence of hypermethylation of VSX2 and an absence of hypermethylation of SEPT9 can still indicate a high likelihood of a colorectal cancer. In other embodiments, a method includes measuring in a sample the methylation status of both GATM and VSX2, wherein hypermethylation of the GATM and the VSX2 indicates a high likelihood of a colorectal cancer. In some aspects, detecting a presence of hypermethylation of GATM and hypermethylation of VSX2 and an absence of hypermethylation of SEPT9 can still indicate a high likelihood of a colorectal cancer.

In some embodiments, the panel of marker genetic loci further comprise SEPT9.

In various aspects, the methylation analysis, methylation level, and reference methylation level all refer to CpG methylation status.

In various aspects, the methylation level is a percentage (%) of methylated cytosines, wherein methylated and unmethylated cytosines make up for 100%. In some aspects, the methylation level is a percentage of methylated cytosines in CpG dinucleotide sites of a genetic locus with genomic coordinates as disclosed herein. In further aspects, the methylation level is a percentage of methylated cytosines in CpG dinucleotide sites in promoter region of the one or more marker genes.

In some embodiments, methylation of the one or more marker genetic loci are methylation of one of the following: (1) EMBP1, (2) DNM1P46, (3) EMBP1 and DNM1P46, (4) EMBP1 and SEPTIN9, (5) DNM1P46 and SEPTIN9, (6) EMBP1, DNM1P46, and SEPTIN9, (7) VSX2, (8) GATM, (9) MAP3K14-AS1, (10) VSX2 and GATM, (11) VSX2 and MAP3K14-AS1, (12) GATM and MAP3K14-AS1, (13) VSX2, GATM, and MAP3K14-AS1, (14) VSX2 and SEPTIN9, (8) GATM and SEPTIN9, (9) MAP3K14-AS1 and SEPTIN9, (10) VSX2, GATM, and SEPTIN9, (11) VSX2, MAP3K14-AS1, and SEPTIN9, (12) GATM, MAP3K14-AS1, and SEPTIN9, or (13) VSX2, GATM, MAP3K14-AS1, and SEPTIN9.

In further embodiments, methylation occurs, or is measured, at hg38 coordinates described in Table 2 of the (marker) genetic loci. “Target regions” of the coordinates of the hg38 assembly are shown in Table 2, for which the methylation-specific real-time PCR assays, Methy Light, were designed in the Examples for cfDNA testing, and they showed peak levels of methylation in stage I/II colorectal cancer tumor).

In yet other embodiments, methylation occurs, or is measured, in extended genetic loci compared to the hg38 coordinates shown in Table 2. The “target regions” of Table 2 are located within a more extended, full CpG island:
EMBP1 CpG island range: Chr1:121519060-121519727 (668 bp),
DNM1P46 CpG island range: Chr15:99806520-99807249 (730 bp),
GATM CpG island range: Chr15:45377773-45378831 (1059 bp),
VSX2 CpG island range: Chr14:74239486-74241489 (2004 bp),
LAYN CpG island range Chr11:111540208-111541474 (1267 bp),
MAP3K14-AS1 CpG island range: Chr17:45261748-45262475 (728 bp), and
SFMBT2 CpG island range: Chr10:7407415-7413250 (5,836 bp).

We conceive that applicable methylation sequencing assays can be designed for these extended genetic loci, which contains regions outside of the “target regions” shown in Table 2, and the assays can still find high levels of methylation which are signature and indicative of CRC. As such, in various embodiments, the ctDNA markers of the present invention are not confined to the “target regions,” and can be located more broadly within the CpG islands shown above. Additionally, the cg probes from the 450k array data in The Cancer Genome Atlas, shown in the Examples section, were located within these full CpG islands, not necessarily within the “target regions” of Table 2. Therefore, measuring a methylation level of a genetic locus, e.g., EMBP1, can refer to measuring a methylation level within a CpG island range Chr1:121519060-121519727, or in some instances more specifically, within Chr1:121519493-121519559, for the EMBP1 genetic locus.

In various embodiments, methylation of EMBP1 refers to methylation in a CpG island range between hg38 coordinates Chr1:121519060-121519727. Hence, detection of hypermethylation of EMBP1 is, or includes, detection of a higher methylation level in CpG island range between hg38 coordinates Chr1:121519060-121519727 in a test sample relative to a reference methylation level, or relative to a methylation level in the same CpG island range in a reference sample.

In further embodiments, methylation of EMBP1 refers to methylation in a CpG island range at least between hg38 coordinates Chr1:121519493-121519559. Hence, detection of hypermethylation of EMBP1 is, or includes, detection of a higher methylation level in a CpG island range at least between hg38 coordinates Chr1:121519493-121519559 in a test sample relative to a reference methylation level, or relative to a methylation level in that CpG island range in a reference sample.

In various embodiments, methylation of DNM1P46 refers to, methylation in a CpG island range between hg38 coordinates Chr15:99806520-99807249. Hence, detection of hypermethylation of DNM1P46 is, or comprises, detection of a higher methylation level in a CpG island range between hg38 coordinates Chr15:99806520-99807249 in a test sample relative to a reference methylation level, or relative to a methylation level in the same CpG island range in a reference sample.

In further embodiments, methylation of DNM1P46 includes methylation in a CpG island range at least between hg38 coordinates Chr15:99806584-99806722. Hence, detection of hypermethylation of DNM1P46 is, or comprises, detection of a higher methylation level in a CpG island range at least between hg38 coordinates Chr15:99806584-99806722 in a test sample relative to a reference methylation level, or relative to a methylation level in that CpG island range in a reference sample.

In various embodiments, methylation of GATM refers to methylation in a CpG island range between hg38 coordinates Chr15:45377773-45378831. Hence, detection of hypermethylation of GATM is, or includes, detection of a higher methylation level in CpG island range between hg38 coordinates Chr15:45377773-45378831 in a test sample relative to a reference methylation level, or relative to a methylation level in the same CpG island range in a reference sample.

In further embodiments, methylation of GATM includes methylation in a CpG island range at least between hg38 coordinates Chr15:45378270-45378365. Hence, detection of hypermethylation of GATM is, or includes, detection of a higher methylation level in a CpG island range at least between hg38 coordinates Chr15:45378270-45378365 in a test sample relative to a reference methylation level, or relative to a methylation level in that CpG island range in a reference sample.

In various embodiments, methylation of VSX2 refers to methylation in a CpG island range between hg38 coordinates Chr14:74239486-74241489. Hence, detection of hypermethylation of VSX2 is, or includes, detection of a higher methylation level in CpG island range between hg38 coordinates Chr14:74239486-74241489 in a test sample relative to a reference methylation level, or relative to a methylation level in the same CpG island range in a reference sample.

In further embodiments, methylation of VSX2 includes methylation in a CpG island range at least between hg38 coordinates Chr14:45378270-74240641. Hence, detection of hypermethylation of VSX2 is, or includes, detection of a higher methylation level in a CpG island range at least between hg38 coordinates Chr14:45378270-74240641 in a test sample relative to a reference methylation level, or relative to a methylation level in that CpG island range in a reference sample.

In various embodiments, methylation of LAYN refers to methylation in a CpG island range between hg38 coordinates Chr11:111540208-111541474. Hence, detection of hypermethylation of LAYN is, or includes, detection of a higher methylation level in CpG island range between hg38 coordinates Chr11:111540208-111541474 in a test sample relative to a reference methylation level, or relative to a methylation level in the same CpG island range in a reference sample.

In further embodiments, methylation of LAYN includes methylation in a CpG island range at least between hg38 coordinates chr11:111540870-111541174. Hence, detection of hypermethylation of LAYN is, or includes, detection of a higher methylation level in a CpG island range at least between hg38 coordinates chr11:111540870-111541174 in a test sample relative to a reference methylation level, or relative to a methylation level in that CpG island range in a reference sample.

In various embodiments, methylation of MAP3K14-AS1 refers to methylation in a CpG island range between hg38 coordinates chr17:45261748-45262475. Hence, detection of hypermethylation of MAP3K14-AS1 is, or includes, detection of a higher methylation level in CpG island range between hg38 coordinates chr17:45261748-45262475 in a test sample relative to a reference methylation level, or relative to a methylation level in the same CpG island range in a reference sample.

In further embodiments, methylation of MAP3K14-AS1 includes methylation in a CpG island range at least between hg38 coordinates Chr17:45262243-45262339. Hence, detection of hypermethylation of MAP3K14-AS1 is, or includes, detection of a higher methylation level in a CpG island range at least between hg38 coordinates Chr17:45262243-45262339 in a test sample relative to a reference methylation level, or relative to a methylation level in that CpG island range in a reference sample.

In various embodiments, methylation of SFMBT2 includes methylation in a CpG island range at least between hg38 coordinates 7407415-7413250. Hence, detection of hypermethylation of SFMBT2 is, or includes, detection of a higher methylation level in a CpG island range at least between hg38 coordinates Chr10:7407415-7413250 in a test sample relative to a reference methylation level, or relative to a methylation level in that CpG island range in a reference sample.

In further embodiments, methylation of SFMBT2 includes methylation in a CpG island range at least between hg38 coordinates Chr10:7410510-7410579. Hence, detection of hypermethylation of SFMBT2 is, or includes, detection of a higher methylation level in a CpG island range at least between hg38 coordinates Chr10:7410510-7410579 in a test sample relative to a reference methylation level, or relative to a methylation level in that CpG island range in a reference sample.

In various embodiments, methylation of SEPT9 refers to methylation in a CpG loci of cg20275528. Hence, detection of the presence of mSEPT9 is, or includes, detection of a higher methylation level in cg20275528 in a test sample relative to a reference methylation level, or relative to a methylation level in the same loci in a reference sample.

In various aspects, the biological sample is a cell-free DNA (cfDNA) sample. In some aspects, the biological sample is obtained from blood or plasma. In some embodiments, the biological sample is obtained from stool or a fecal specimen. In some embodiments, the biological sample is a formalin-fixed paraffin-embedded tissue. In other embodiments, the biological sample is a frozen sample, e.g., fresh frozen sample. In various aspects, the biological sample is pre-treated for bisulfite-conversion of the marker genes.

In some embodiments, the above-identified methylation markers, or assays thereof, are used in a subject desiring a result, such as age 30 years old or above, age 35 year old or above, age 40 years old or above, age 45 years old or above, age 50 years old or above, age 55 years old or above, age 60 years old or above, age 70 years old or above, age 80 years old or above, or age 90 years old or above. The subject may be asymptomatic of colorectal cancer. In further embodiments, the above-identified methylation markers, or assays thereof, are used in a subject having undergone surgery or a neoadjuvant systemic therapy for colorectal cancer. The methylation status of the marker genetic loci can be used to predict or detect recurrence of colorectal disease, especially after a prior treatment or surgery.

In some embodiments, the above-identified biomarkers, or assays thereof, are used for early detection of colorectal cancer from a biological sample of a subject, especially one who is unable or refusing to undergo a colonoscopy. In some embodiments, the above-identified biomarkers, or assays thereof, are used to triage a subject in need of a colonoscopy. In some embodiments, the above-identified biomarkers, or assays thereof, are used in a subject who has not undergone a colonoscopy.

In some embodiments, the above-identified biomarkers, or assays thereof, are used for detection of the presence of “minimal residual disease”, “molecular residual disease” or disease recurrence following surgical resection of a solid tumor, or in rectal cancer patients undergoing neoadjuvant chemo-radiotherapy prior to surgery. The persistence of ctDNA in plasma after surgical resection provides a surrogate for the presence of residual disease and has been correlated with poor prognosis and is predictive of cancer recurrence (relapse). CtDNA testing for minimal residual disease can provide an indication of the adequacy of the surgical resection in disease intervention. In some embodiments, the subject in need of an assay described herein is one having had surgical resection of the colon, and the assay detects biomarkers indicative of colorectal cancer recurrence or poor prognosis.

In some embodiments, the above-identified methylation markers, or assays thereof, are used for early prediction or detection of colorectal cancer recurrence. e.g., especially those recurrence after treatment with curative intent such as surgery or neoadjuvant systemic therapy. The subsequent re-emergence of positive plasma ctDNA signals during post-operative surveillance is a harbinger of cancer recurrence. This detection of either “molecular residual disease” or the re-emergence of positive ctDNA signals in serial plasma samples from blood drawn post-operatively may precede the detection of metastatic lesions by standard-of-care radiological imaging by up to two years. This is referred to as the “lead time” from ctDNA detection to radiological detection of metastasis. Importantly, the detection of molecular residual disease or recurrence can guide oncologists about treatment decision-making, for example the intensification of adjuvant (post-operative) chemotherapy. In some embodiments, further treatment is administered to a subject detected with a hypermethylation in one or more of the marker genetic loci disclosed herein, based on understanding that hypermethylation in one or more of the marker genetic loci indicates a likelihood of colorectal cancer reoccurrence or presence of residual colorectal cancer tissue/disease.

In some embodiments, the above-identified biomarkers, or assays thereof, are used for predicting progression of early-stage (stage I or II) CRC to stage IV or III. In some embodiments, the above-identified biomarkers are detected in biological samples from subjects with a colon cancer or rectal cancer. In some embodiments, the above-identified biomarkers are detected in biological samples from subjects at stage I colorectal cancer. In some embodiments, the above-identified biomarkers are detected in biological samples from subjects at stage II colorectal cancer. In some embodiments, the above-identified biomarkers are detected in biological samples from subjects at stage I or II colorectal cancer. In some embodiments, the above-identified biomarkers are also detected in biological samples from subjects at stage III or IV colorectal cancer. In some embodiments, the above-identified biomarkers are detected in biological samples from subjects at stage III colorectal cancer. In some embodiments, the above-identified biomarkers are detected in biological samples from subjects at stage IV colorectal cancer.

In some embodiments, the above-identified biomarkers, or assays thereof, are used for detecting, predicting, or monitoring colorectal cancer disease progression or response to treatment in a subject with a colorectal cancer. In some embodiments, the above-identified biomarkers, or assays thereof, are used for detecting circulating tumor cells, and the detected presence of which is prognostic of poor clinical outcome of the subject.

In some embodiments, the above-identified biomarkers, or assays thereof, are used for predicting substantially complete clinical and/or pathological response to, or monitoring tumor burden in response to, a systemic therapy, a neoadjuvant chemotherapy, or a radiation therapy, e.g., in a subject with localized rectal cancer or stage IV colorectal cancer.

In the setting of metastatic disease, ctDNA levels have been shown to correlate with tumor volume. Serial ctDNA testing may allow for the intensification, change, or sequencing of neoadjuvant (presurgical)systemic therapies and the tumor response to these therapies. In some embodiments, the subject in need of an assay described herein is a colorectal cancer subject having had a neoadjuvant chemotherapy or radiation therapy.

In some embodiments, the above-identified biomarkers, or assays thereof, are used for screening for colorectal cancer (e.g., colon cancer). For example, a method of screening for colorectal cancer comprises performing colonoscopy on a subject measured or detected, in a biological sample from the subject, with a methylation level of one or more marker genetic loci above a reference methylation level, wherein the marker genetic loci are or include (genes and/or pseudogenes) DNM1P46, EMBP1, GATM, VSX2, MAP3K14-AS1, LAYN, SFMBT2, or a combination thereof, and optionally further including SEPT9. In some embodiments, a method of screening for colon cancer comprises performing colonoscopy on subject measured or detected, in a biological sample from the subject, with a methylation level of one or more marker genetic loci above a reference methylation level, wherein the marker genetic loci include or are DNM1P46, EMBP1, GATM, VSX2, MAP3K14-AS1, LAYN, SFMBT2, or a combination thereof, and optionally further including SEPT9.

Various embodiments provide a method for treating a subject with colorectal cancer, which includes providing a treatment to a subject measured or detected in a biological sample of the subject with a methylation level of one or more marker genetic loci above a reference methylation level. The one or more marker genetic loci include or are DNM1P46, EMBP1, GATM, VSX2, MAP3K14-AS1, LAYN, SFMBT2, or a combination thereof, and optionally further including SEPT9.

Further embodiments provide a method for treating a subject with colorectal cancer, and the method includes measuring a methylation level of one or more marker genes in a biological sample of the subject, and providing a treatment to the subject if the methylation level of the one or more marker genes in the biological sample is above a reference level. The one or more marker genetic loci include or are DNM1P46, EMBP1, GATM, VSX2, MAP3K14-AS1, LAYN, SFMBT2, or a combination thereof, and optionally further including SEPT9.

Other embodiments provide a method for treating a subject with colorectal cancer, and the method includes requesting the measurement of a methylation level of one or more marker genetic loci in a biological sample of the subject, wherein the one or more marker genetic loci comprise DNM1P46, EMBP1, GATM, VSX2, MAP3K14-AS1, LAYN, SFMBT2, or a combination thereof, and optionally further comprising SEPT9, and providing a treatment to the subject if the methylation level of the one or more marker genes in the biological sample is above a reference level.

Further embodiments provide methods for identifying and treating a subject detected with the biomarkers disclosed herein indicative of colorectal cancer, wherein the treatment includes a local treatment, a systemic treatment, or both.

Local treatments treat the tumor without affecting the rest of the body. Exemplary local treatments include surgery for colon cancer, surgery for rectal cancer, ablation and embolization for colorectal cancer, and radiation therapy for colorectal cancer. In various implementations, local treatments are performed in subjects with earlier stage cancers (or smaller cancers that haven't spread). Chemotherapy can be given to a region, such as through hepatic artery infusion, which can be used for colorectal cancer that has spread to the liver.

Systemic treatments can reach cancer cells throughout almost all the body. Colorectal cancer can also be treated using drugs, which can be given by mouth or directly into the bloodstream. Exemplary systemic treatments for colorectal cancer include chemotherapy (e.g., 5-fluorouracil, Capecitabine, Irinotecan. Oxaliplatin, Triflundine and tipiracil), targeted therapy (e.g., drugs that target blood vessel formation: Bevacizumab, Ramucirumab, Ziv-aflibercept; drugs that target cancer cells with EGFR changes: cetuximab, panitumumab; drugs that target cells with BRAF gene changes: encorafenib: drugs that are kinase inhibitor regorafenib), and immunotherapy (e.g., PD-1 inhibitors: pembrolizumab, nivolumab: CTLA-4 inhibitors: ipilimumab). Chemotherapy can be administered at different times, for example, adjuvant chemo is given after surgery, and neoadjuvant chemo is given before surgery. Chemo drugs for colon or rectal cancer that are given into a vein (IV), can be given either as an injection over a few minutes or as an infusion over a longer period of time. Exemplary neoadjuvant therapy involves radiotherapy, chemotherapy used alone or in combination. Exemplary chemotherapy agents include 5-fluorouracil (5-FU) and oxaliplatin.

In some embodiments, treatment of colon cancer is selected by stage of the cancer. For example, treating stage 0 colon cancer is surgery to take out the cancer. Treatment stage I colon cancer includes colonoscopy removal, or surgical removal of the section of colon that has cancer and nearby lymph nodes. Treating stage II colon cancer may include surgical removal and adjuvant chemotherapy. Treating stage III colon cancer may include surgical removal followed by adjuvant chemo, and possibly neoadjuvant chemotherapy along with radiation to shrink the cancer so as to facilitate removal by surgery. Treating stage IV colon cancer may include chemo therapy, and possibly surgery to relieve symptoms of the cancer. Another option after initial chemotherapy might be treatment with an immunotherapy drug and/or radiation.

In some embodiments, treatment of rectal cancer is selected by stage of the cancer. For example, treating stage 0 rectal cancer may include removal or destroying the cancer via surgery, such as a polypectomy, local excision, or transanal resection. Treating stage I rectal cancer may include removal during colonoscopy or surgery. Treating stage II rectal cancer may include chemotherapy, radiation therapy, and possibly further with surgery. Treating stage III rectal cancer may include chemotherapy, radiation therapy, and surgery, such as chemo and radiation before surgery, then surgery, then further chemo; or chemo alone first, followed by chemo plus radiation, then followed by surgery. Treating stage IV rectal cancer may include surgery, chemo, chemoradiation.

Also provided are assays or methods for measuring methylation level or performing methylation analysis. We have developed highly sensitive real-time methylation-specific PCR-based assays for the detection of CRC signals in bisulfite-converted cell-free DNA (cfDNA) in blood plasma for selected markers that were discovered in our pipeline. These assays are designed to be “short”, and are therefore readily detected in samples where the DNA is fragmented, including cell-free DNA (cfDNA) from blood plasma, stool, and formalin-fixed paraffin-embedded (FFPE) tissue. The marker coordinates could similarly be used for the detection of CRC ctDNA signals using deep next-generation sequencing based methods on bisulfite-converted cfDNA.

In some embodiments, a real-time methylation-specific PCR-based assay is used for the detection of colorectal cancer signals in a biological sample obtained from a subject having or suspected of having colorectal cancer. These assays can be used to detect samples where the DNA is fragmented, and are highly sensitive for identification of colorectal cancer when indicted methylation markers are detected present.

In various implementations, measuring the methylation level comprises:
a) treating DNA in the biological sample with one or more reagents to convert unmethylated cytosine bases to uracil sulfonate or another base having a different binding behavior than cytosine, while methylated cytosine bases remain unchanged;
b) amplifying the treated DNA in the presence of a forward primer oligonucleotide and a reverse primer oligonucleotide, and optionally a polymerase, wherein each of the forward primer oligonucleotide and the reverse primer oligonucleotide hybridizes specifically onto the treated DNA of the one or more marker genes; and
c) deducing percentage of methylated cytosine bases in the amplified DNA in step b) of the one or more marker genes as the methylation level.

In further aspects, the amplified DNA in step b) is fewer than 91 bp in length, preferably no more than 90 bp in length, or 50-54, 55-60, 61-65, 66-70, 71-74, 75-80, 81-85, or 86-90 bp in length, and in various instances at least 35, 36, 37, 38, 39, 40, 41, 42, 43, 44, 45, 46, 47, 48, 49, or 50 bp or longer in length. In further aspects, the first and the second oligonucleotides are independently 20, 21, 22, 23, 24, 25, 26, 27, 28, 29 or 30 bases in length (e.g., 23-29 bp or 26 bp), and suitable for use as primers for the one or more marker genetic loci. Preferably, the first and/or the second oligonucleotides hybridize onto at least one (e.g., 1, 2, 3, 4, 5, or more) CpG dinucleotide sites in the marker genetic locus. In some aspects, the first and/or the second oligonucleotides hybridize onto the promoter region of the marker genetic locus, including at least one or at least two CpG dinucleotide sites. In some embodiments, the first and the second oligonucleotides are a forward PCR primer and a reverse PCR primer disclosed in Table 2, or an oligonucleotide sequence that has deletion of 1, 2, 3, 4, or 5 nucleotides compared to the forward PCR primer sequence or reverse PCR primer sequence disclosed in Table 2.

In some aspects, the amplification is in the presence of a third oligonucleotide, which hybridizes onto a third region of the marker gene(s), and the third region may include at least three or two CpG dinucleotide sites. The third oligonucleotide may be modified with a detectably labeled moiety, such as a fluorescent moiety, an illuminescent moiety, an enzymatic moiety, a chemiluminescent moiety, a nanodot, or a nanoparticle. In some aspects, the third oligonucleotide is about 22, 23, 24, 25, 26, 27, 28, 29, or 30 bases in length. In some aspects, the region(s) of the marker gene(s) bound by the first and/or the second oligonucleotide overlap(s) the region bound by the third oligonucleotide; and/or the amplified DNA is no more than 90 bp in length. In some aspects, the amplification in the presence of the third oligonucleotide allows for real-time deduction of presence of methylated cytosine bases in the amplified DNA. In some embodiments, the third oligonucleotide is a Probe sequence disclosed in Table 2, or an oligonucleotide sequence that has deletion of 1, 2, 3, 4, or 5 nucleotides compared to the Probe sequence of Table 2.

In some aspects, treating DNA in the biological sample with one or more reagents to obtain bisulfite-converted DNA is a treatment with a bisulfite reagent or solution, such as sodium bisulfite, or with a reagent that permits sulfonation, deamination, or desulfonation on the DNA. In other aspects, treating DNA in the biological sample with one or more reagents is a treatment with a cytidine deaminase. Cytidine deaminases convert unmethylated cytidine faster than methylated cytidine. In various aspects, treating DNA in the biological sample with one or more reagents results in bisulfite-converted cell-free DNA (cfDNA) in the biological sample.

In some aspects, deducing percentage of methylated cytosine bases is based on methylation sequencing, including bisulfite sequence PCR (BSP), methylation specific PCR (MSP), MethyLight, methylation-sensitive high resolution melting (MS-HRM), or next-generation sequencing (NGS).

Primers for methylation sequencing of the one or more marker genes can be designed on software platforms such as Methyl Primer Express (Applied Biosystems, Foster City, CA), MethPrimer, BiSearch, MethMaker, and MSPprimer. Further details of methylation sequencing and primer designs are described in BioTechniques, vol. 55, no. 4, 2018, which is incorporated by reference herein in its entirety.

Further embodiments provide the one or more marker genetic loci measured, selected, or assayed is any one of DNM1P46, EMBP1, GATM, VSX2, MAP3K14-AS1, LAYN, and SFMBT2. In some embodiments, the one or more marker genetic loci measured as having a higher methylation level than a reference methylation level is any one of DNM1P46, EMBP1, GATM, VSX2, MAP3K14-AS1, LAYN, and SFMBT2. In some embodiments, the one or more marker genetic loci measured, selected, or assayed includes any one of DNM1P46, EMBP1, GATM, VSX2, MAP3K14-AS1, LAYN, and SFMBT2. In some embodiments, the one or more marker genetic loci measured as having a higher methylation level than a reference methylation level includes any one of DNM1P46, EMBP1, GATM, VSX2, MAP3K14-AS1, LAYN, and SFMBT2. In some embodiments, the one or more marker genetic loci measured, selected, or assayed are SEPT9 and any one of DNM1P46, EMBP1, GATM, VSX2, MAP3K14-AS1, LAYN, and SFMBT2. In some embodiments, the one or more marker genetic loci measured as having a higher methylation level than a reference methylation level are SEPT9 and any one of DNM1P46, EMBP1, GATM, VSX2, MAP3K14-AS1, LAYN, and SFMBT2. In some embodiments, the one or more marker genetic loci measured, selected, or assayed include SEPT9 and any one of DNM1P46, EMBP1, GATM, VSX2, MAP3K14-AS1, LAYN, and SFMBT2. In some embodiments, the one or more marker genetic loci measured as having a higher methylation level than a reference methylation level includes SEPT9 and any one of DNM1P46, EMBP1, GATM, VSX2, MAP3K14-AS1, LAYN, and SFMBT2.

In some embodiments, the one or more marker genetic loci measured, selected, or assayed is EMBP1. In some embodiments, the one or more marker genetic loci measured as having a higher methylation level than a reference methylation level is EMBP1. In some embodiments, the one or more marker genetic loci measured, selected, or assayed includes EMBP1. In some embodiments, the one or more marker genetic loci measured as having a higher methylation level than a reference methylation level includes EMBP1.

In some embodiments, the one or more marker genetic loci measured, selected, or assayed is DNM1P46. In some embodiments, the one or more marker genetic loci measured as having a higher methylation level than a reference methylation level is DNM1P46. In some embodiments, the one or more marker genetic loci measured, selected, or assayed includes DNM1P46. In some embodiments, the one or more marker genetic loci measured as having a higher methylation level than a reference methylation level includes DNM1P46.

In some embodiments, the one or more marker genetic loci measured, selected, or assayed is GATM In some embodiments, the one or more marker genetic loci measured as having a higher methylation level than a reference methylation level is GATM. In some embodiments, the one or more marker genetic loci measured, selected, or assayed includes GATM. In some embodiments, the one or more marker genetic loci measured as having a higher methylation level than a reference methylation level includes GATM.

In some embodiments, the one or more marker genetic loci measured, selected, or assayed is VS2. In some embodiments, the one or more marker genetic loci measured as having a higher methylation level than a reference methylation level is VSX2. In some embodiments, the one or more marker genetic loci measured, selected, or assayed includes VSX2. In some embodiments, the one or more marker genetic loci measured as having a higher methylation level than a reference methylation level includes VSX2. In further embodiments, measuring a higher methylation level than a reference methylation level in VSX2 indicates the subject has a stage I colorectal cancer.

In some embodiments, the one or more marker genetic loci measured, selected, or assayed is LAYN. In some embodiments, the one or more marker genetic loci measured as having a higher methylation level than a reference methylation level is LAYN. In some embodiments, the one or more marker genetic loci measured, selected, or assayed includes LAYN. In some embodiments, the one or more marker genetic loci measured as having a higher methylation level than a reference methylation level includes LAYN.

In some embodiments, the one or more marker genetic loci measured, selected, or assayed is MAP3K14-AS1. In some embodiments, the one or more marker genetic loci measured as having a higher methylation level than a reference methylation level is MAP3K14-AS1. In some embodiments, the one or more marker genetic loci measured, selected, or assayed includes MAP3K14-AS1. In some embodiments, the one or more marker genetic loci measured as having a higher methylation level than a reference methylation level includes MAP3K14-AS1.

In some embodiments, the one or more marker genetic loci measured, selected, or assayed is SFMBT2. In some embodiments, the one or more marker genetic loci measured as having a higher methylation level than a reference methylation level is SFMBT2. In some embodiments, the one or more marker genetic loci measured, selected, or assayed includes SFMBT2. In some embodiments, the one or more marker genetic loci measured as having a higher methylation level than a reference methylation level includes SFMBT2.

In further embodiments, the one or more marker genetic loci measured, selected, or assayed are two or more of DNM1P46. EMBP1, GATM, VSX2, MAP3K4-AS1, LAYN, and SFMBT2. In some embodiments, the one or more marker genetic loci measured as having a higher methylation level than a reference methylation level are two or more of DNM1P46, EMBP1, GATM, VSX2, MAP3K14-AS1, LAYN, and SFMBT2. In some embodiments, the one or more marker genetic loci measured, selected, or assayed include two or more of DNM1P46, EMBP1, GATM, VSX2, MAP3K14-AS1, LAYN, and SFMBT2. In some embodiments, the one or more marker genetic loci measured as having a higher methylation level than a reference methylation level include two or more of DNM1P46, EMBP1, GATM, VSX2, MAP3K14-AS1, LAYN, and SFMBT2.

In some embodiments, the one or more marker genetic loci measured, selected, or assayed are EMBP1 and DNM1P46. In some embodiments, the one or more marker genetic loci measured as having a higher methylation level than a reference methylation level are EMBP1 and DNM1P46.

In further embodiments, the one or more marker genetic loci measured, selected, or assayed are SEPT9 and any two or more of DNM1P46, EMBP1, GATM, VSX2, MAP3K14-AS1, LAYN, and SFMBT2. In some embodiments, the one or more marker genetic loci measured as having a higher methylation level than a reference methylation level are SEPT9 and any two or more of DNM1P46, EMBP1, GATM, VS-2, MAP3K14-AS1, LAYN, and SFMBT2. In some embodiments, the one or more marker genetic loci measured, selected, or assayed include SEPT9 and any two or more of DNM1P46, EMBP1, GATM, VSX2, MAP3K14-AS1, LAYN, and SPMBT2. In some embodiments, the one or more marker genetic loci measured as having a higher methylation level than a reference methylation level includes SEPT9 and any two or more of DNM1P46, EMBP1, GATM, VSX2 MAP3K14-AS1, LAYN and SFMBT2.

In some embodiments, the one or more marker genetic loci measured, selected, or assayed, or measured as having a higher methylation level than respective reference methylation levels, are SEPTIN9 and both of EMBP1 and DNM1P46. In some embodiments, the one or more marker genetic loci measured, selected, or assayed, or measured as having a higher methylation level than respective reference methylation levels, are SEPTIN9 and EMBP1. In some embodiments, the one or more marker genetic loci measured, selected, or assayed, or measured as having a higher methylation level than respective reference methylation levels, are SEPTIN9 and DNM1P46.

In further embodiments, the one or more marker genetic loci measured, selected, or assayed are three or more of DNM1P46, EMBP1, GATM, VSX2, MAP3K14-AS1, LAYN and SFMBT2. In some embodiments, the one or more marker genetic loci measured as having a higher methylation level than a reference methylation level are three or more of DNM1P46, EMBP1, GATM, VS2, MAP3K14-AS1, LAYN, and SFMBT2. In some embodiments, the one or more marker genetic loci measured, selected, or assayed include three or more of DNM1P46, EMBP1, GATM, VSX2, MAP3K14-AS1, LAYN, and SFMBT2. In some embodiments, the one or more marker genetic loci measured as having a higher methylation level than a reference methylation level include three or more of DNM1P46, EMBP1, GATM, VSX2, MAP3K4-AS1, LAYN, and SFMBT2.

In some embodiments, the one or more marker genetic loci measured, selected, or assayed, or measured as having a higher methylation level than respective reference methylation levels, are VSX2, GATM, and MAP3K14-AS1. In some embodiments, the one or more marker genetic loci measured, selected, or assayed, or measured as having a higher methylation level than respective reference methylation levels, are any two of VSX2, GATM, and MAP3K14-AS1. In some embodiments, the one or more marker genetic loci measured, selected, or assayed, or measured as having a higher methylation level than respective reference methylation levels, are VS2 and GATM. In some embodiments, the one or more marker genetic loci measured, selected, or assayed, or measured as having a higher methylation level than respective reference methylation levels, are VSX2 and MAP3K14-AS1. In some embodiments, the one or more marker genetic loci measured, selected, or assayed, or measured as having a higher methylation level than respective reference methylation levels, are GATM and MAP3K14-AS1.

In further embodiments, the one or more marker genetic loci measured, selected, or assayed are SEPT9 and any three or more of DNM1P46, EMBP1, GATM, VSX2, MAP3K14-AS1, LAYN, and SFMBT2. In some embodiments, the one or more marker genetic loci measured as having a higher methylation level than a reference methylation level are SEPT9 and any three or more of DNM1P46, EMBP1, GATM, VSX2, MAP3K14-AS1, LAYN, and SFMBT2. In some embodiments, the one or more marker genetic loci measured, selected, or assayed include SEPT9 and any three or more of DNM1P46, EMBP1, GATM, VSX2, MAP3K14-AS1, LAYN, and SFMBT2. In some embodiments, the one or more marker genetic loci measured as having a higher methylation level than a reference methylation level includes SEPT9 and any three or more of DNM1P46, EMBP1, GATM, VSX2, MAP3K14-AS1, LAYN, and SFMBT2.

In some embodiments, the one or more marker genetic loci measured, selected, or assayed, or measured as having a higher methylation level than respective reference methylation levels, are SEPTIN9, VSX2, GATM, and MAP3K14-AS1. In some embodiments, the one or more marker genetic loci measured, selected, or assayed, or measured as having a higher methylation level than respective reference methylation levels, are SEPTIN9 and any one or two of VSX2, GATM, and MAP3K14-AS1. In some embodiments, the one or more marker genetic loci measured, selected, or assayed, or measured as having a higher methylation level than respective reference methylation levels, are SEPTIN9, VSX2 and GATM. In some embodiments, the one or more marker genetic loci measured, selected, or assayed, or measured as having a higher methylation level than respective reference methylation levels, are SEPTIN9, VSX2 and MAP3K14-AS1. In some embodiments, the one or more marker genetic loci measured, selected, or assayed, or measured as having a higher methylation level than respective reference methylation levels, are SEPTIN9, GATM and MAP3K14-AS1.

Additional embodiments provide that the one or more marker genetic loci measured, selected, or assayed are any four or more of DNM1P46, EMBP1, GATM, VSX2, MAP3K14-AS1, LAYN, and SFMBT2. In some embodiments, the one or more marker genetic loci measured as having a higher methylation level than a reference methylation level are any four or more of DNM1P46, EMBP1, GATM, VSX2, MAP3K14-AS1, LAYN, and SFMBT2. Some embodiments provide that the one or more marker genetic loci measured, selected, or assayed include four or more of DNM1P46, EMBP1, GATM, VSX2, MAP3K14-AS1, LAYN, and SFMBT2. In some embodiments, the one or more marker genetic loci measured as having a higher methylation level than a reference methylation level include four or more of DNM1P46, EMBP1, GATM, VSX2, MAP3K14-AS1, LAYN, and SFMBT2.

In other embodiments, the one or more marker genetic loci measured, selected, or assayed are SEPT9 and any four or more of DNM1P46, EMBP1, GA 7M, VSX2, MAP3K14-AS1, LAYN, and SFMBT2. In some embodiments, the one or more marker genetic loci measured as having a higher methylation level than a reference methylation level are SEPT9 and any four or more of DNM1P46, EMBP1, GATM, VSX2, MAP3K14-AS1, LAYN, and SFMBT2. Some embodiments provide that the one or more marker genetic loci measured, selected, or assayed include SEPT9 and four or more of DNM1P46, EMBP1, GATM, VSX2. MAP3K14-AS1, LAYN, and SFMBT2. In some embodiments, the one or more marker genetic loci measured as having a higher methylation level than a reference methylation level include SEPT9 and four or more of DNM1P46, EMBP1, GATM, VSX2, MAP3K14-AS1, LAYN, and SFMBT2.

Additional embodiments provide that the one or more marker genetic loci measured, selected, or assayed are any five or more of DNM1P46, EMBP1, GATM, VSX2, MAP3K14-AS1, LAYN, and SFMBT2. In some embodiments, the one or more marker genetic loci measured as having a higher methylation level than a reference methylation level are any five or more of DNM1P46, EMBP1, GATM, VSX2, MAP3K14-AS1, LAYN, and SFMBT2. Some embodiments provide that the one or more marker genetic loci measured, selected, or assayed include five or more of DNM1P46, EMBP1, GATM, VSX2, MAP3K14-AS1, LAYN, and SFMBT2. In some embodiments, the one or more marker genetic loci measured as having a higher methylation level than a reference methylation level include five or more of DNM1P46, EMBP1, GATM, VSX2, MAP3K14-AS1, LAYN, and SFMBT2.

In other embodiments, the one or more marker genetic loci measured, selected, or assayed are SEPT9 and any five or more of DNM1P46, EMBP1, GATM, VSX2, MAP3K14-AS1, LAYN, and SFMBT2. In some embodiments, the one or more marker genetic loci measured as having a higher methylation level than a reference methylation level are SEPT9 and any five or more of DNM1P46, EMBP1, GATM, VSX2, MAP3K14-AS1, LAYN, and SFMBT2. Some embodiments provide that the one or more marker genetic loci measured, selected, or assayed include SEPT9 and five or more of DNM1P46, EMBP1, GATM, VSX2, MAP3K14-AS1, LAYN, and SFMBT2. In some embodiments, the one or more marker genetic loci measured as having a higher methylation level than a reference methylation level include SEPT9 and five or more of DNM1P46, EMBP1, GATM, VSX2, MAP3K14-AS1, LAYN, and SFMBT2.

Additional embodiments provide that the one or more marker genetic loci measured, selected, or assayed are any six or more of DNM1P46, EMBP1, GATM, VSX2, MAP3K14-AS1, LAYN, and SPMBT2. In some embodiments, the one or more marker genetic loci measured as having a higher methylation level than a reference methylation level are any six or more of DNM1P46. EMBP1, GA 7M, VSX2, MAP3K14-AS1, LAYN, and SFMBT2. Some embodiments provide that the one or more marker genetic loci measured, selected, or assayed include six or more of DNM1P46, EMBP1, GATM, VSX2, MAP3K14-AS1, LAYN, and SFMBT2. In some embodiments, the one or more marker genetic loci measured as having a higher methylation level than a reference methylation level include six or more of DNM1P46, EMBP1, GATM, VSX2, MAP3K14-AS1, LAYN, and SFMBT2.

In other embodiments, the one or more marker genetic loci measured, selected, or assayed are SEPT9 and any six or more of DNM1P46, EMBP1, GATM, VSX2, MAP3K14-AS1, LAYN, and SFMBT2. In some embodiments, the one or more marker genetic loci measured as having a higher methylation level than a reference methylation level are SEPT9 and any six or more of DNM1P46, EMBP1, GATM, VSX2, MAP3K14-AS1, LAYN, and SFMBT2. Some embodiments provide that the one or more marker genetic loci measured, selected, or assayed include SEPT9 and six or more of DNM1P46, EMBP1, GATM, VSX2, MAP3K14-AS1, LAYN, and SFMBT2. In some embodiments, the one or more marker genetic loci measured as having a higher methylation level than a reference methylation level include SEPT9 and six or more of DNM1P46, EMBP1, GATM, VSX2, MAP3K14-AS1, LAYN, and SFMBT2.

Additional embodiments provide that the one or more marker genetic loci measured, selected, or assayed are all seven of DNM1P46, EMBP1, GATM, VSX2, MAP3K14-AS1, LAYN, and SFMBT2. In some embodiments, the one or more marker genetic loci measured as having a higher methylation level than a reference methylation level are all seven of DNM1P46, EMBP1, GATM, VSX2, MAP3K14-AS1, LAYN, and SFMBT2. Some embodiments provide that the one or more marker genetic loci measured, selected, or assayed include all seven of DNM1P46, EMBP1, GATM, VSX2, MAP3K14-AS1, LAYN, and SFMBT2. In some embodiments, the one or more marker genetic loci measured as having a higher methylation level than a reference methylation level include all of DNM1P46, EMBP1, GATM, VSX2, MAP3K4-AS1, LAYN, and SFMBT2.

In other embodiments, the one or more marker genetic loci measured, selected, or assayed are SEPT9 and all of DNM1P46, EMBP1, GATM, VSX2, MAP3K14-AS1, LAYN, and SFMBT2. In some embodiments, the one or more marker genetic loci measured as having a higher methylation level than a reference methylation level are SEPT9 and all of DNM1P46, EMBP1, GATM, VSX2, MAP3K14-AS1, LAYN, and SFMBT2. Some embodiments provide that the one or more marker genetic loci measured, selected, or assayed include SEPT9 and all of DNM1P46, EMBP1, GATM, VSX2, MAP3K14-AS1, LAYN, and SFMBT2. In some embodiments, the one or more marker genetic loci measured as having a higher methylation level than a reference methylation level include SEPT9 and all of DNM1P46, EMBP1, GATM, VSX2, MAP3K14-AS1, LAYN, and SFMBT2.

In further embodiments, the one or more marker genetic loci measured, selected, or assayed for methylation status are combined with genetic mutations and/or proteins in a combinatorial panel to detect CRC.

Our panel of methylation markers could be applied for the detection and/or semi-quantification of CRC signals in samples of tissue, blood, or stool for multiple scenarios. This may include, but is not limited to, the following applications:
1. Early detection of CRC from a blood or stool sample when applied to population-based screening of screen-eligible adults (e.g., those unable/refusing to undergo a colonoscopy), or to triage those in need of a colonoscopy.
2. Detection of minimal residual disease (MRD) or disease recurrence in CRC patients following surgical resection with curative intent, to determine if surgery was complete, or in rectal cancer patients undergoing neoadjuvant chemo-radiotherapy prior to surgery. The detection of MRD/recurrence after surgery is a predictor of disease recurrence and poor prognosis.
3. Prediction of complete clinical and complete pathological response to neoadjuvant chemo and/or radiation therapy (e.g., localized rectal cancer or stage IV CRC).
4. Prediction/detection of disease progression (e.g., in stage IV CRC cases).
5. Monitoring responses to treatment.
6. Detection of circulating tumor cells, which is prognostic of poor outcome.

In some embodiments, methods are provided for detecting and/or classifying a colorectal carcinoma or colorectal cell proliferative disorder in a subject, wherein the methods include:
contacting DNA from a biological sample obtained from a human subject with at least one agent that provides for determination of a CpG methylation status of one or more genes of DNM1P46, EMBP1, GATM, VSX2, MAP3K14-AS, LAYN, and SFMBT2, optionally further including SEPT9;
determining, based on said contacting, a CpG methylation status of the one or more genes; and
detecting and/or classifying a colorectal carcinoma or colorectal cell proliferative disorder in the subject based on increased CpG methylation of the one or more genes, relative to that of a reference methylation level.

In some embodiments, methods are also for detecting CpG dinucleotide methylation in cfDNA of one or more genes of DNM1P46, EMBP1, GATM, VSX2, MAP3K14-AS1, LAYN, and SFMBT2, optionally further including SEPT9. The methods include extracting or otherwise isolating cfDNA from a biological sample obtained from a subject; treating the cfDNA, or a fragment thereof comprising the one or more genes, with one or more reagents to convert cytosine bases that are unmethylated in the 5-position thereof to uracil or to another base that is detectably dissimilar to cytosine in terms of hybridization properties; contacting the treated cfDNA, or the treated fragment thereof, with an amplification enzyme and at least one oligonucleotide or peptide nucleic acid (PNA) oligomer comprising a contiguous sequence of at least 5, 6, 7, 8, 9, 10, 11, 12, 13, 14, or 15 nucleotides that is identical to, complementary to, or hybridizes to a sequence selected from those whose hg38 coordinates are provided in Table 2, and the complements thereof, wherein the treated cfDNA or the fragment thereof is amplified to produce at least one amplificon; and detecting by determining a presence of, or a property of, the at least one amplificon, the methylation state or level of at least one CpG dinucleotide of the one or more genes, or an average, or a value reflecting an average methylation state or level of a plurality of CpG dinucleotides of the one or more genes.

In further embodiments, treating the cfDNA or fragment thereof comprises use of a reagent that is bisulfite, hydrogen sulfite, disulfite, and any combinations thereof.

In further embodiments, contacting DNA includes contacting the DNA with at least one reagent, or series of reagents that distinguishes between methylated and non-methylated CpG dinucleotides within at least one target region of the DNA, wherein the target region comprises, or hybridizes to, a sequence of at least 10, at least 13, at least 16, at least 20, or at least 30, contiguous nucleotides of at least one sequence selected from those whose hg38 coordinates are provided in Table 2, wherein said contiguous nucleotides comprise at least one CpG dinucleotide sequence.

In further embodiments, a neoplastic colorectal cell proliferative disorder is colorectal cancer. In various aspects, the neoplastic colorectal cell proliferative disorder is metastases of colorectal cancer. In further implementations, the subject has colorectal cancer or a colorectal cell proliferative disorder, and the method is carried out repeatedly.

Further embodiments provide that the one or more methylation markers (also referred to as ctDNA markers) disclosed herein are used for assessing efficacy or effectiveness of treatment to a subject with CRC. Further embodiments provide that the one or more methylation markers (also referred to as ctDNA markers) disclosed herein are used for monitoring progression of CRC in a subject, or in a subject undergoing a treatment. Further embodiments provide that the one or more methylation markers (also referred to as ctDNA markers) disclosed herein are used for determining CRC tumor burden in a subject. In various aspects, the subject being assessed for CRC treatment effectiveness, monitored for progression, or determined for tumor burden is one with metastatic CRC. In other aspects, the subject being monitored for CRC progression or regression, or determined for tumor burden, is one without a CRC treatment.

In various implementations of these methods, if the levels of these methylation markers go up, it indicates or predicts progression of the CRC; if the levels of these methylation markers go down, it indicates or predicts responsiveness to a treatment; and if the levels of these methylation markers remain stable, it indicates that the CRC stable (or in oncology the CRC is considered under good control). Usually, the levels are compared to those measured before for the determination of levels going up, going down, or remaining stable. For example, an assessment after a treatment is compared to that before the treatment, or compared to that in an earlier time after the start of the treatment.

In some embodiments, methods are provided for monitoring progression of the colorectal cancer in the subject, (or for assessing efficacy or effectiveness of a treatment to a subject with colorectal cancer.) and the methods include:
measuring a methylation level of one or more marker genetic loci in a first biological sample obtained from the subject at a time t0,
measuring a methylation level of the one or more marker genetic loci in a second biological sample obtained from the subject at a time t1, said time t1 being subsequent to said time t0, (and for assessing the efficacy or effectiveness of the treatment said time t1 being subsequent to the treatment,
wherein the one or more marker genetic loci comprise any one, two, three, four, five, six, or all of genes of: DNM1P46, EMBP1, GATM, VSX2, MAP3K14-AS1, LAYN, and SFMBT2, and optionally further comprising SEPT9, and
wherein the colorectal cancer is indicated to show regression or has not worsened, (or the treatment is indicated to be effective) when DNM1P46, EMBP1, GATM, VSX2, MAP3K14-AS1, LAYN, SFMBT2, and SEPT9, if selected, each has a lower methylation level in the second biological sample obtained at the time t1 relative to that in the first biological sample obtained at the time t0;
wherein the colorectal cancer is indicated to have worsened, (or the treatment is indicated to require intensification) when DNM1P46, EMBP1, GATM, VSX2, MAP3K14-AS1, LAYN, SFMBT2, and, SEPT9, if selected, each has a higher methylation level in the second biological sample obtained at the time t1 relative to that in the first biological sample obtained at the time t0.

In additional implementations, an indication of CRC progression is followed with intensification of treatment. Intensification of treatment is conducted at the discretion of the treating oncologist, as this is associated with reduced overall survival time, which may include increasing drug dosing, treatment time, and/or adding additional treatment. In additional implementations, an indication of stable CRC or responsiveness to a treatment is followed with continuation of existing treatment, or no change in the treatment regimen, as treatment is indicated to be effective.

Types of treatment for CRC are mentioned in preceding paragraphs. For example, treatment can be one or more of: colonoscopy removal, or surgical removal of the section of colon that has cancer and nearby lymph nodes; chemotherapy, adjuvant chemotherapy, or neoadjuvant chemotherapy; radiation; and/or immunotherapy.

### Kits/Compositions

Some embodiments provide a kit containing reagents, or reagents and controls samples, for methylation analysis of a biological sample, preferably liquid-based biological sample containing cfDNA.

In some embodiments, a kit comprises at least a first isolated nucleic acid which hybridizes onto a first region of a marker gene (or an amplified fragment thereof) selected from DNM1P46, EMBP1, GATM, VSX2, MAP3K14-AS1, LAYN, SFMBT2, a combination of DNM1P46, EMBP1, GATM, VSX2, MAP3K4-AS1, LAYN, and SFMBT2, and a combination of SEPT9 and one or more of DNM1P46, EMBP1, GATM, VSX2, MAP3K4-AS1, LAYN, and SFMBT2; and at least a second isolated nucleic acid which hybridizes onto a second region of the marker gene: wherein the first and the second isolated nucleic acids are suitable for use as primers.

In some embodiments, a kit further comprises a third isolated nucleic acids modified with a detectable label (detectably labeled moiety), a quencher, or both, wherein the third isolated nucleic acid hybridizes onto a third region of the marker gene (or an amplified fragment thereof).

In some embodiments, a kit comprises a bisulfite reagent, a container suitable for containing said bisulfite reagent and a biological sample of a patient, and at least one set of oligonucleotides containing two or more oligonucleotides whose sequences in each case are identical, are complementary, or hybridize to a 9 or more, preferably 18 base long or longer, segment of a sequence selected from those whose hg38 coordinates are provided in Table 2.

In some aspects, the first and the second isolated nucleic acids are oligonucleotides, each about 22-30 bases in length and capable of hybridizing at least one, two, or three CpG dinucleotide sites. In some aspects, the third isolated nucleic acid is an oligonucleotide about 22-30 bases in length and capable of hybridizing at least two or three CpG dinucleotide sites.

In some embodiments, the first, second, and/or third isolated nucleic acids are bound to a solid phase. In other embodiments, the kit further comprises a solid phase support, in addition to the first, second, and/or third isolated oligonucleotides. The nucleic acid molecules, or oligonucleotides, may constitute all or part of an “array” or “DNA chip”. The solid-phase surface may be composed of silicon, glass, polystyrene, aluminium, steel, iron, copper, nickel, silver, or gold. Nitrocellulose as well as plastics such as nylon, which can exist in the form of pellets or also as resin matrices, may also be used.

In further embodiments, the kit additionally comprises a control sample containing respective marker gene(s), including amplified fragment thereof, obtained from normal colon mucosa or from a biological sample from a control subject free of colorectal cancer.

Some embodiments provide a composition comprising an isolated nucleic acid molecule or its isolated fully complementary nucleic acid molecule, and (i) an entity selected from the group consisting of chromophores, fluorophores, lipids, cholic acid, thioethers, aliphatic chains, phospholipids, polyamines and polyethylene glycol, wherein the isolated nucleic acid molecule or the fully complementary nucleic acid molecule is chemically linked to the entity, or (ii) a solid phase support, wherein the isolated nucleic acid molecule or the fully complementary nucleic acid molecule is bound to the solid phase support, and wherein the isolated nucleic acid molecule has a sequence comprising at least 10, at least 13, at least 16, at least 20, or at least 30 contiguous nucleotides of a nucleic acid sequence selected from those whose hg38 coordinates are provided in Table 2.

Further description of reagents in a kit or reagents and techniques suitable for methylation analysis is provided in U.S. Pat. No. 8,623,599 and US Patent Application Publication No. 20200131582, which are incorporated by reference in their entirety.

### Examples

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Study Design

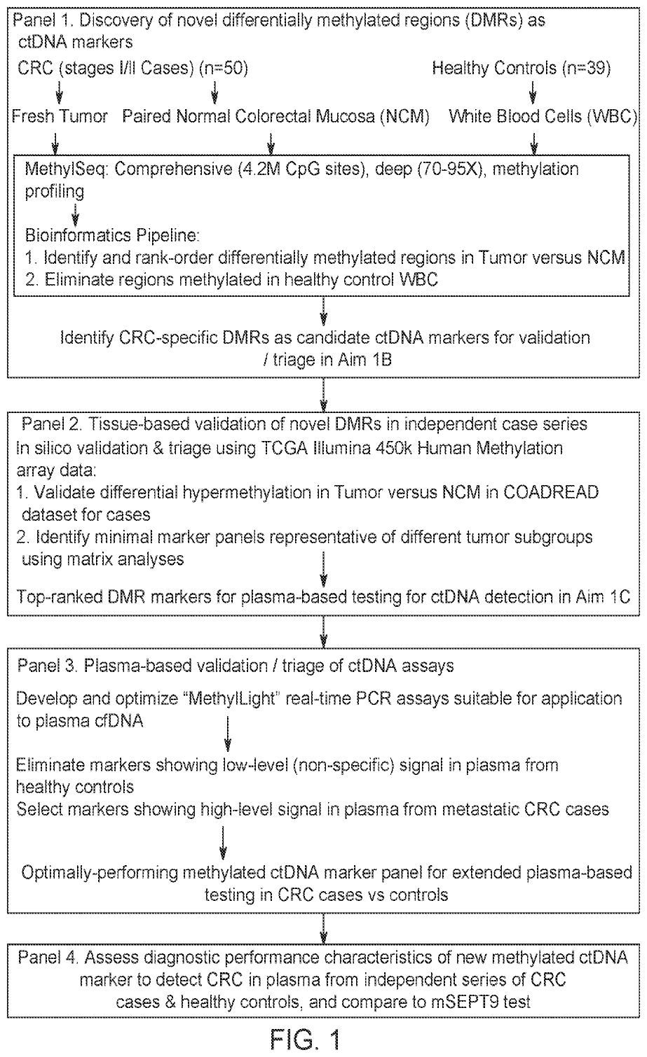

FIG. 1 shows the design of a study of the paired early-stage CRC tumors and distant normal colorectal mucosa (NCM) tissue from patients diagnosed with stage I/II colorectal cancer. For the biomarker discovery (Panel 1 of FIG. 1), we identified regions of DNA that were hypermethylated in early-stage tumor tissues, had low/no methylation in the distant NCM, and were unmethylated in the white blood cells (WBC)/leukocytes of healthy persons. The reasoning for this was: (1) Markers that are hypermethylated in tumors would shed DNA into the bloodstream, which would be detectable as circulating tumor DNA (ctDNA) within the cell-free DNA (cfDNA) of the blood plasma. Hypermethylated DNA biomarkers are more readily detectable via highly sensitive methylation-specific PCR assays, which could provide inexpensive and rapid-turnaround end-point assays for future clinical testing. This type of assay would detect the presence of aberrant cancer-specific methylation at these genomic loci as the specific cancer signal itself. (2) Methylated DNA is more nuclease resistant than unmethylated DNA, so is a more stable analyte in plasma (which has high levels of nucleases), and would therefore produce more robust signals if cancer was present. (3) Given plasma cfDNA in healthy controls is derived mainly from WBC, it was important to filter out any non-specific methylation that is naturally present in WBC in the normal state. This would ensure the methylated markers identified were likely to be specific to CRC-derived ctDNAs within the plasma cfDNA and would not give rise to false-positive results in people without cancer, cfDNA (including ctDNA if present) is highly fragmented. Therefore, a ctDNA detection test in plasma would require design using methylation-specific real-time PCR assays that were as short as possible in length (ideally ≤72 bp) to produce amplicons when ctDNA is present. Therefore, the bioinformatics biomarker discovery pipeline was also designed to search for short regions containing methylated CpG sites that were amenable to this type of assay.

For tissue-based validation (Panel 2 of FIG. 1), we used methylation data obtained on the Infinium, HumanMethylation 450k array (Illumina) platform, from CRC tumor and paired adjacent NCM tissue available to researcher through. The Cancer Genome Atlas (TCGA), colon and rectal adenocarcinoma (COADREAD) dataset. Promising markers that “validated” would then proceed to assay design for application to ctDNA detection in blood plasma, and these would then be tested in a small number of plasma samples from patients with colorectal cancer and healthy (non-cancer) control subjects (Panel 3 of FIG. 1).

Finally, for assays that validated in initial plasma samples, a more extensive assessment of the diagnostic performance of the biomarkers would be conducted in a sufficiently-powered case-control study of CRC cases and non-cancer controls (Panel 4 of FIG. 1). The mSEPT9 (Epi proColon V2.0 test) would be used as a “yardstick” to assess the performance and redundancy or complementarity of our new biomarkers.

Numerous statistical analyses were performed to assess biomarker prevalence, synergy, and complementarity to identify minimal panels of complementary markers that had high collective sensitivity to detect tumors. Human genome coordinates were provided of the biomarkers identified from the bioinformatics pipeline. The biomarkers were validated in bioinformatics-based interrogations of the 450k array methylation data from the COADREAD dataset of The Cancer Genome Atlas (TCGA). The methylation-specific real-time PCR (MethyLight) assays were also designed and optimized for the detection of the methylated ctDNA markers with high analytical sensitivity and specificity for application to cfDNA from plasma, for the blood-based detection of CRC signals.

### Source of Tissue Samples for Biomarker Discovery

Paired tumor-normal colorectal mucosa (NCM) tissues: Samples of the colorectal cancer (tumor) tissue and distant NCM tissue (taken around 10 cM away from the tumor margin to avoid contamination with tumor cells) were shipped on dry ice. Limited demographic data and relational molecular pathology data included the tumor features of BRAF V600E and KRAS codons 12 and 13 mutation status, microsatellite instability (MSI) status, CpG island methylator phenotype (CIMP) status, and MLH1, MGMT, and P16 (CDNK2A) methylation status, as described in Diagn Mol Pathol 2009; 18:62-71, Mod Pathol 2009; 22:1588-99, and Mod Pathol 2011; 24:396-411. These data were used to ensure tumors representative of the distinct molecular subtypes of CRC were included in the discovery set, and were later used for orthogonal analyses of the new methylation biomarkers to determine if the new biomarkers were biased towards any one molecular phenotype of CRC.

Blood samples for white blood cells (WBC) and plasma: Plasma-based validation and assessment of diagnostic performance characteristics of the methylated ctDNA markers were obtained from CRC patients undergoing treatment (colonoscopy, surgery, chemo- and radiation oncology) and from patients without CRC undergoing a procedure (e.g. colonoscopy or surgery for hemorrhoids) as non-cancer controls. Blood samples from non-cancer controls were also used to obtain WBC for Methyl-Seq. These samples were collected under an Institutional Review Board-approved protocol, Pro00054104.

Methyl-Seq: Quantification and quality control of the DNA samples was performed using the Qubit and Nanodrop spectrophotometer. Some tumor-NCM DNA samples with low concentration were omitted. The platform used for Methyl-Seq was the SureSelect^{XT} Methyl-Seq Target Enrichment System for Illumina Multiplexed Sequencing (Agilent Technologies) to capture and sequence CpG-rich regions including about 4.2 million individual CpG sites, following a protocol described in www.agilent.com/cs/library/usermanuals/public/G7530-90002.pdf. This platform encompasses all the CpG sites included in the Human Methylation 450k and EPIC array-based methods, and more, including CpG islands, enhancers, and other types of regulatory elements that are involved in gene regulation. Next-generation sequencing (NGS) of the Methyl-Seq libraries was performed to ˜70×-95× average depth, although this was somewhat variable in the tumor and NCM samples, depending on DNA concentration.

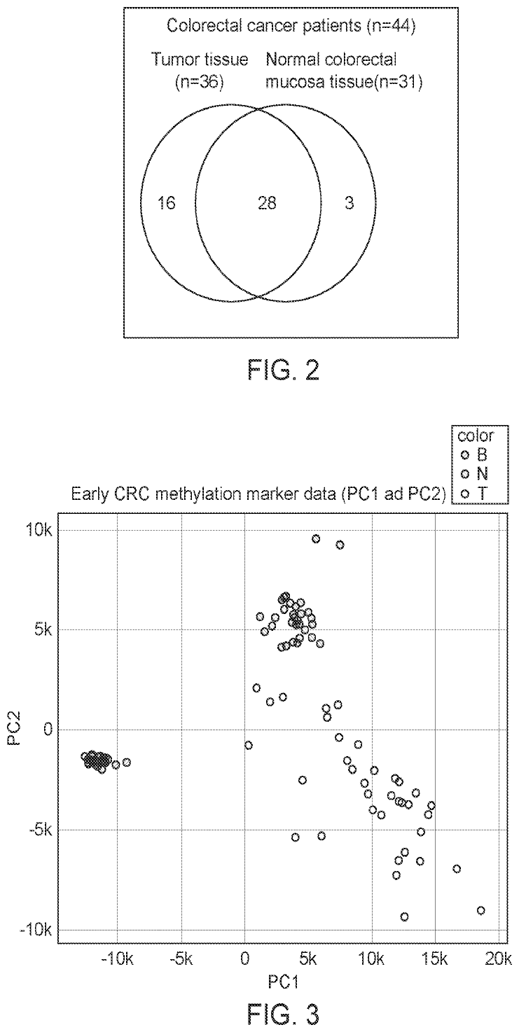

High-quality Methyl-Seq data was obtained from fresh-frozen stage I/II CRC and/or paired NCM tissue samples from 44 CRC cases aged 41-91 years (mean 71 years), of which 28 cases had paired samples of tumor and NCM (36 had tumor, 31 had NCM) (FIG. 2). We also included WBC from 39 healthy controls to filter out non-specific markers, given WBC are the major contributing cell type to normal cfDNA. After various quality control (QC) checks on the raw data, unsupervised clustering of the methylomes and principal components analysis (PCA) showed the samples segregated into three groups by tissue-type, and the tumor tissues were more variable (FIG. 3).

Bioinformatics analyses of the Methyl-Seq data: This tailored bioinformatics pipeline first mapped the Methyl-Seq NGS reads (FASTQ files) to the human genome, and thereafter, sought differentially methylated regions (DMRS). The parameters we used to define these DMRs were regions hypermethylated in tumors with mean and median >30% higher methylation compared to the NCM samples (to take into account the NCM from patients could harbor low-level methylation as a field cancerization effect), and <0.5% mean and <0.1% median methylation level in WBC from healthy controls. To derive these DMRs we took into account the clinical endpoint application for their detection as ctDNA in plasma using methylation-specific MethyLight real-time PCR assays. Given that most cfDNA is fragmented into mononucleosomal size fragments of ≤167 bp, PCR-based assays of short amplicon size (<100 bp) have shown an improved rate of detection of ctDNA. Furthermore, for the design of MethyLight assays, each oligonucleotide (forward and reverse PCR primers and fluorescence-labeled reporter probe in-between) needs to span 1-3 differentially-methylated CpG sites. (See Table 2. We thus applied a sliding window approach to search for ≤72 bp DMRs containing 7-16 CpG sites that would be amenable for the design of MethyLight assays. From this, we identified and rank-ordered the highly significant short DMRs, which included several markers for which plasma- or fecal-based assays have previously been developed for CRC detection, namely SEPTIN9, VIM, C9ORF50, BCAT1, and ITGA4. The re-identification of these markers lends confidence to our discovery pipeline.

Lasso regression and PCA were then applied to identify non-redundant short DMRs that were independent of tumor molecular features, including KRAS and BRAF mutations, microsatellite instability (MSI), CIMP, and MGMT methylation. (FIG. 4).

Validation of the Methylated DNA Markers in Methylation Data from the Human Genome Atlas:

Next, the Infinium HumanMethylation 450k array (Illumina) data from the Colon and Rectum Adenocarcinoma (COADREAD) dataset from The Cancer Genome Atlas (TCGA) were explored to determine the consistency of differential methylation for our new biomarkers in an independent series of tumor and adjacent NCM samples from CRC cases. Very few of the CpG sites contained within our new DMRs were represented on the 450k array, which is one possible reason why these markers may not have previously been identified; Methyl-Seq data are much higher-resolution (4.2 million CpGs) than the 450k array (˜450,000 CpGs). Thus, we were unable to use the 450k array data from TCGA to directly validate all of our DMRs. However, we were able to explore the CpG site(s) represented on the 450k array located closest to (≤250 bp), and within the same CpG-rich feature, for most of our top-ranked DMRs. These individual CpG sites showed highly significant differences in the levels of methylation in primary CRC tissues versus paired normal tissues (but not always recurrent or metastatic tissues) in the COADREAD dataset. See FIGS. 4B-4L for illustrative data for a subset of the markers. Not every new DMR we identified had a CpG probe in the 450k array data in TCGA located within the same CpG feature; for example, the highly-ranked methylated (m) DNM1P46 (Dynamin 1 Pseudogene 46) marker was not represented on the 450k array. For these markers, no tissue-based validation was performed. Instead, these proceeded directly for validation in “spare” plasma samples from metastatic CRC cases, for which plasma was available from multiple serial blood draws, and the mSEPT9 ctDNA status was known.

Assessment of Complementarity to mSEPT9:

The complementarity to each candidate marker was assessed by assessing the correlation of methylation levels of individual markers with each of the other markers. See FIG. 4M for a heatmap feature correlation. As examples, this correlation heatmap shows low correlation between the new MAP3K14-AS1, GATM, and VSX2 markers and mSEPT9, indicating that these three markers may complement mSEPT9 (i.e. are more frequently methylated in tumors where mSEPT9 is unmethylated/lost by genetic mechanisms).

Design and Optimization of “MethyLight” (Methylation-Specific Real-Time PCR Assays) with High Analytical Sensitivity and Specificity for Application to Plasma-Based Detection of ctDNA

Design: For the detection of methylation as the CRC signal in plasma cfDNA, the template cfDNA will be first bisulfite-converted, hence assays are designed to amplify the bisulfite-converted template cfDNA. Bisulfite-conversion coverts unmethylated cytosines to uracil, whilst methylated cytosines at CpG dinucleotide sites are inert to this treatment and remain as cytosines within the sequence. This allows for appropriately-designed PCR assays to differentiate markers based on their methylation status/content. Other groups have shown that PCR-based assays of short amplicon size (<100 bp) have improved rate of detection of ctDNA within plasma cfDNA. However, these assays were designed to detect tumor-derived “somatic” sequence mutations within plasma cfDNA, and not for bisulfite-converted templates. For the detection of methylated ctDNA, given the cfDNA is first chemically converted with bisulfite, this can further degrade the cfDNA template into even smaller fragments.

The Assay was designed as “in silico” conversion of the original DNA sequence to sequences that would be produced when fully methylated (CpG dinucleotides sites retained as CpG) or unmethylated (CpG dinucleotide sites converted to UpG), whilst all isolated cytosines are converted to uracil. PCR primers were designed to incorporate 2-3 differential CpG sites, with a nested fluorescent labeled probe spanning at least 3 differentially methylated and contiguous CpG sites. These assays were designed to be a maximum length of 90 bp, which would allow for spacing of PCR amplification primers (˜26 bp each), plus the fluorescent-labeled probe (˜26 bp) to span CpG sites (see Table 2). Assays of shorter length were feasible for some markers, but this was dependent on sequence content and spacing of the CpG sites. In the case of assays at 56-65 bp in length, there was some overlap in the CpG sites utilized in both the PCR primer at one end of the target region and the fluorescent-labeled probe, providing the primer and probe bound to opposite (sense or antisense) strands, hence neither competed for binding to template nor interfered with the reaction efficiency (too little overlap to form oligonucleotide dimers). In summary, each MethyLight qPCR assay was designed for endpoint application for the sensitive and specific detection of low copy-number, fragmented, methylated ctDNA in plasma. These assays are run simultaneously with the exact same ACTB reaction as that used in the mSEPT9® assay in a 2-color duplex reaction, or a 3-color triplex reaction (i.e., two test markers can be combined), with ACTB and the test marker(s) labeled with distinct fluorescent probes. ACTB serves as a control for the amount and integrity of plasma cfDNA template input into the PCR reaction (which may, or may not, contain ctDNA). We opted to use the same ACTB PCR as used in the mSEPT9 test for cross-comparison of our new methylated ctDNA markers with mSEPT9 performance. In our initial optimizations and early plasma tests, we used CY5 as the fluorescent label for ACTB and FAM or HEX (hexachloro-fluorescein) for each methylated ctDNA marker, as these fluoresceins lie in separate light emission spectra, with no interference between them.

Improvement: The qPCR primer annealing temperatures were fine tuned using bisulfite-converted DNA from the RKO CRC cell line (hypermethylated template) versus leukocyte DNA from a healthy control subject (this should be unmethylated template) with SYBR Green on annealing temperature gradients followed by PCR met curves using the CFX 96 thermal cycling system (Bio-Rad). This was to establish the optimal annealing temperature at which the qPCR was efficient and methylation-specific, and indicate homogeneity of the PCR products using the melt curve. This was then repeated with the probe included within a reduced temperature range (without the melt curve), and thereafter, with ACTB included for a duplex reaction. For some markers, high signals were obtained in the leukocyte DNA template, either indicating the PCR was not methylation-specific, or indicating methylation was present in the leukocyte samples. Depending on the findings, either a new assay was designed, or the marker was omitted as non-specific. Assays from different markers that had similar thermal cycling conditions could be combined to make a triplex assay (two new markers+ACTB control).

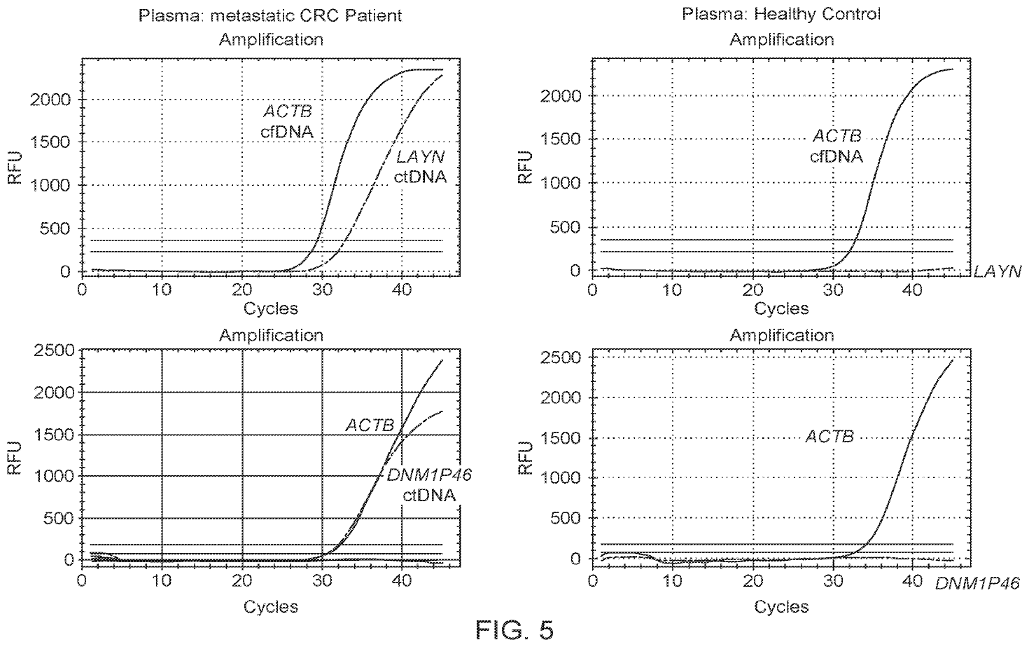

Testing in plasma: The duplex assays were then tested on a limited number of cfDNA extracts (bisulfite-converted) from plasma of metastatic CRC (mCRC) cases and plasma from healthy controls to verify there was high signal levels in mCRC plasma and no signal in healthy control plasma, as illustrated in FIG. 5. A few assays were eliminated at this point due to the consistent detection of low-level signal in plasma from healthy controls, consistent with the presence of methylation within a normal cell type(s) that contribute to the cfDNA pool, and which are detectable using our high-sensitivity assays. By this stage, we had eliminated OPLAH, AMER2, GATA3, HLA-V, KCNA1, ACTR3C, and PPPR1R16B.

For assays that demonstrated (1) efficient qPCR “MethyLight” amplification. (2) high level signal in plasma from mCRC cases, and (3) absent signal in plasma from healthy controls, we then proceeded to extend the testing to additional mCRC patients to determine which markers were positive most frequently and were non-redundant. We eliminated a few markers at this point due to redundancy (i.e. they are positive only in a subset of mCRC cases for which other markers were more frequently positive). E.g. we eliminated LAYN, EVL, and SFMBT2, as redundant. We may re-evaluate these in early-stage CRC cases in future, as these markers were validated in primary (but not metastatic) CRC cases.

Validation of the New Methylated ctDNA Markers Alongside mSEPT9

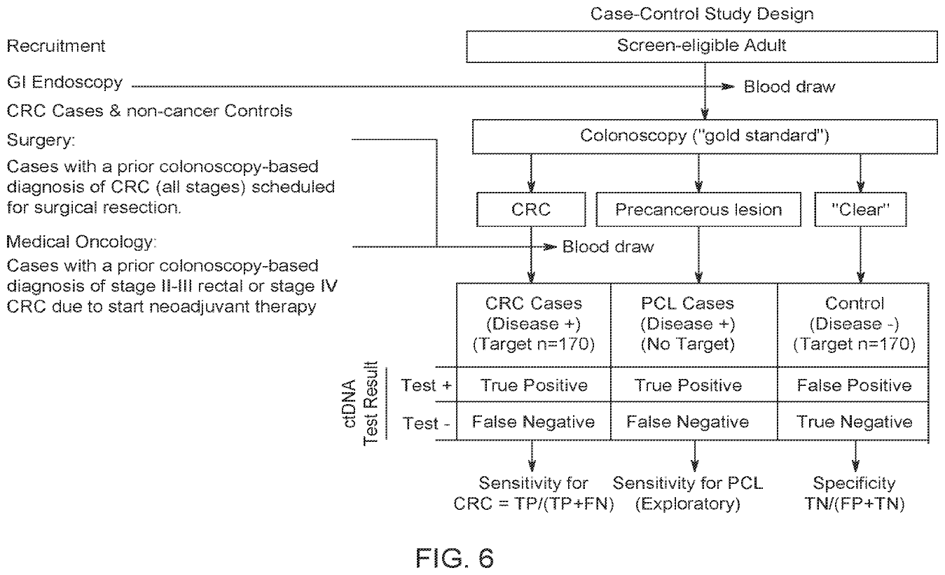

We tested our selected markers alongside mSEPT9 to determine their sensitivity, specificity, and potential complementarity to mSEPT9 in plasma from all-stage (I-IV) CRC cases (to assess sensitivity) and healthy controls (to assess specificity) according to the study designed in FIG. 6. It is important to note that these plasma samples are from an independent series of CRC patients than those used in the Discovery series of patients from Australia (i.e., they are from different patients). This is important, as using plasma from the same patients as the tissues were derived from can result in statistical “overfitting” of marker sensitivity. These plasma series thus represent a true validation of the performance of these markers in patients derived from a distinct population (i.e., USA).

We have tested the following FOUR markers alongside mSEPT9 in plasma from pre-treatment CRC cases of stages I-IV: DNM1P46, EMBP1, GATM, and VSX2, using the oligonucleotides shown in Table 2. In plasma from 50 CRC cases, we obtained data for most markers from most, but not all, of the cases. A grid showing positivity and negativity for each marker for each patient is provided in Table 3. Markers were considered positive if a signal reached a cycle threshold (Ct value)≤45. Markers were considered negative if no signal was detected and the ACTB input control had a Ct value ≤33. For mSEPT9, the FDA-approved conditions for positive and negative test results were used, which are similar to that above.

Sensitivity of mSEPT9 was 68.0% (34/50) among 50 pre-treatment CRC cases and there was significant overlap in mSEPT9 positivity and positivity for each of our new methylated ctDNA markers. Sensitivity of DNM1P46 was 69.2% (27/39) among 39 cases with valid results, and this marker had significant overlap with mSEPT9 (near perfect correlation). Sensitivity of EMBP1 was 61.7% (29/47) among 47 cases with valid results and positively detected a subset of the CRC cases detected by mSEPT9, but also detected one case that was negative for mSEPT9 and two cases that were negative for DNM1P46. Interestingly, EMBP1 also detected minimal residual disease in post-operative CRC cases ahead of mSEPT9, hence may still be of use in a panel. Probably the two most interesting markers were GATM and VSX2, which both had some degree of complementarity to mSEPT9, although their overall sensitivity was lower. Sensitivity of GATM was just 50.0% (24/48) among 48 cases with valid test results. However, GATM detected an additional 3 CRC cases that were negative for mSEPT9. This would have increased the combined sensitivity of mSEPT9 plus GATM by 6.25%, to about 75.5%. Sensitivity of VSX2 was 65.12% (28/43) among 43 cases with valid test results. Interestingly, VSX2 was also complementary to mSEPT9, also detecting 3 CRC cases that were negative for mSEPT9 (2 of these were also detected by GATM), VSX2 was the only marker to detect a CRC case with stage I disease. EMBP1, GATM and VSX2 combined identified 5 cases that mSEPT9 did not. Therefore, EMBP1, GATM and VSX2 are markers that, although whose overall sensitivity as single markers is suboptimal, appear to add value to mSEPT9. Combined sensitivity for, mSEPT9, GATM, and VSX2 was approximately 77.56% (38/49), although results were not achieved for all 49 cases with GATM and VSX2, so this may be an under-estimate. Notably, this patient series had insufficient samples to analyze MAP3K14-AS1 as well.

Plasma cfDNA was tested for markers MAP3K14-AS1, VSX2, DNM1P46, EMBP1, SFMBT2, alongside SEPTIN9 in patients with advanced colon or rectal cancer undergoing neoadjuvant (pre-surgical) chemotherapy to shrink the tumors for surgical candidacy (Table 4). DNM1P46 detected the very same cases as mSEPT9, VSX2 was able to detect cancer signal in two patients that no other marker (including SEPTIN9) detected (illustrating complementarity), MAP3K14-AS1 also identified one case that mSEPT9 did not. SFMBT2 appeared redundant, detecting a proportion of cases detected by other markers. Testing of six (cancer-free) healthy controls produced true-negative test results for all five markers, indicating good specificity for CRC, whereas mSEPT9 showed non-specificity (false-positive) in one healthy control (Table 4).

Plasma cfDNA (1 mL) was tested for markers GATM and VSX2 (as most complementary to mSEPT9 and capable of combining into a triplex reaction with ACTB to minimize plasma usage) to detect minimal residual disease following surgical resection with curative intent from six patients who subsequently experienced recurrence. These six patients were enrolled in cfDNA clinical testing using the tumor mutation next-generation sequencing (NGS) test, SIGNATERA™ (Natera). Interestingly, mSEPT9, GATM and VSX2 detected minimal residual disease with similar accuracy as the clinical SIGNATERA™ test using just 1 mL plasma for each, mSEPT9 and GATM/VSX2 combined. SIGNATERA was positive in 5/6 patients, mSEPT9 was positive in 4/6, and the single test comprising both GATM and VSX2 markers detected all 6/6 patients.

In terms of specificity, all SIX new markers have shown 100% specificity, but only in small numbers of healthy control subjects so far tested. mSEPT9 yielded one false-positive test result in one colonoscopy-verified healthy control subject, which was correctly negative for the new assays (Table 4).

We revisited the methylation levels in the tumor tissues of our Discovery series to determine the performance of SEPTIN9 and the four new markers, EMBP1, DNM1P46, GATM, and VSX2 individually, and in combination, for early-stage (I/I) tumors (Table 5). For this, we used a threshold level of 25% methylation to call a tumor methylation-positive. SEPTIN9 was methylation-positive in just 48.48% of the stage I/II tumors in our Discovery series. Interestingly, sensitivity of each of the four new markers superseded that of SEPTIN9 in our Discovery series. In combination, the four new marker plus SEPTIN9 have 91.67% to detect stage I/II tumor tissues. This combined panel is therefore likely to increase the sensitivity for detection of early-stage (I/II) CRC in cfDNA over SEPTIN9 alone.

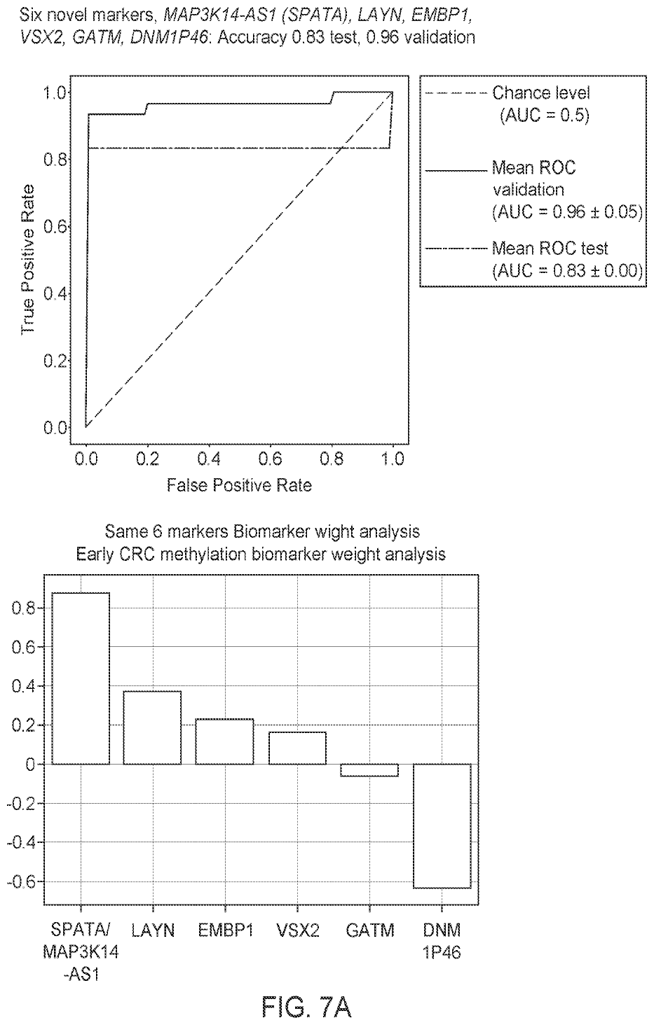

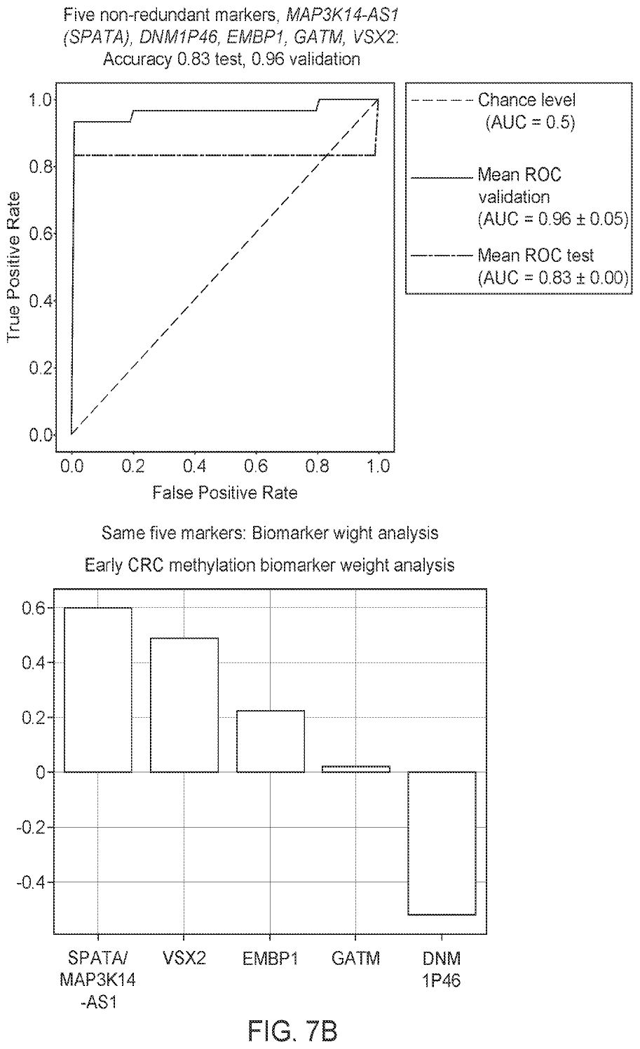

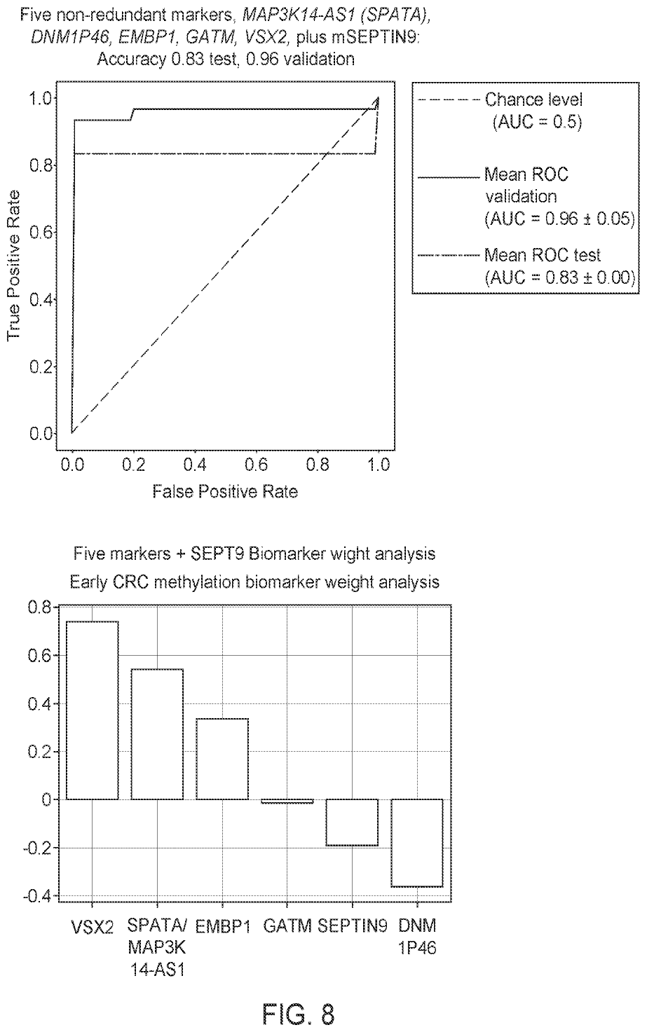

We performed a Machine Learning analysis of the discovery data using logistic regression for six novel markers, MAP3K14-AS1, DNM1P46, EMBP1. VSX2, GATM, and LAYN (redundant in plasma), with and without mSEPT9 (FIGS. 8 and 7, respectively), to determine which markers are most informative and to determine their accuracy to distinguish tumor from normal colorectal mucosa tissue using receiver operating characteristics (ROC) analyses with area under the curve (AUC) to measure accuracy. Without mSEPT9, the accuracy of the 6 markers was 0.96 (validation set) with MAP3K14-AS1 being the most informative (highest value added) marker (FIG. 7A). Accuracy remained the same at 0.96 (validation set) with redundant marker removed and just five non-redundant markers (MAP3K14-AS1. DNM1P46, EMBP1, VSX2, GATM) included (FIG. 7B). With mSEPT9 added to the same five non-redundant markers, the accuracy remained at 0.96 (validation set), VSX2 and MAP3K14-AS/being the two most informative (highest value added) markers (FIG. 8), and DNM1P46 being the least and mSEPT9 being the second least informative markers. These findings are solely based on tissues and may change with increased sample size.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

TABLE 2
Target sequence coordinates of the methylated genetic loci according
to the Hg38 assembly, methylation-specific oligonucleotide sequences,
and thermal cycling conditions for “MethyLight” real-time PCR
amplification of new markers of colorectal cancer.
Probe sequence for each marker is labeled at the 5′-end with a reporter
molecule 6-carboxyfluorescein (/56-FAM/, isomer derivative of fluorescein
attachment) and with a double quencher, ZEN™ Internal Quencher for
TaqMan and qPCR probe(/ZEN/) positioned between the ninth (9th) and
tenth (10th) nucleotide base in the oligonucleotide sequence and IOWA
BLACK® quencher (/31ABKFQ/, suitable for use with fluorescein) located
at the 3′-end. The double-quenched probes generate less background and
increase signal compared to probes containing a single quencher.

| | | | | Probe sequence (5′-3′) | | |
|---|---|---|---|---|---|---|
| | Hg38 | | | (sequence ID) with a | Ampli- | |
| | Coordinates | Forward | Reverse | fluorescent | con | |
| | of target | PCR primer | PCR primer | reporter molecule | size | Tₘ |
| Marker | region | sequence (5′-3′) | sequence (5′-3′) | and a double quencher | (bp) | (° C) |
| EMBP1 | Chr1: | TATAGGGGTTAGGGTAG | CAACCCTACCCGAAACG | 56-FAM/CCTCTACCC/ | 88 | 62 |
| | 121519493- | GGAGGGC | CCAACG | ZEN/GCGAAAACCCGAACC | | |
| | 121519559 | (SEQ ID NO: 1) | (SEQ ID NO: 2) | TC/31ABKFQ | | |
| | | | | (SEQ ID NO: 3, absent labels) | | |
| DNM1P | Chr15: | GAGAGGCGAAGTCGCG | CTATCATAACTACGACC | 56-FAM/CGGGCGGTT/ | 61 | 60 |
| 46 | 99806584- | AGCGTTC | ACCCGACG | ZEN/TCGTCGGGTGGTCGT | | |
| | 99806722 | (SEQ ID NO: 4) | (SEQ ID NO: 5) | AG/3IABKFQ | | |
| | | | | (SEQ ID NO: 6, absent labels) | | |
| GATM | Chr15: | AACACTCGCTCGCTCCC | TAACTCACTCGATCCTA | 56-FAM/AACACTCGC/ | 81 | 60 |
| | 45378270- | TACCCGA | CGAACGACG | ZEN/TCGCTCCCTACCCGA/ | | |
| | 45378365 | (SEQ ID NO: 7) | (SEQ ID NO: 8) | 3IABKFQ | | |
| | | | | (SEQ ID NO: 9. absent labels) | | |
| VSX2 | Chr14: | TTCGAGCGGGTCGCGAT | CTCGAAAACCCTAAATA | 56-FAM/AGTAGCGCG/ | 74 | 60 |
| | 45378270- | ATATTTTTC | ACCTCTCCG | ZEN/GGGAGAAGGCGGAG | | |
| | 74240641 | (SEQ ID NO: 10) | (SEQ ID NO: 11) | /3IABKFQ | | |
| | | | | (SEQ ID NO: 12, absent labels) | | |
| LAYN | chr11: | ATTATTTAGTAGGCGAT | CGCTACAAACCGTACTA | 56-FAM/CTACGAACC/ | 71 | 62 |
| | 111540870- | TCGTCGCG | CTAACCG | ZEN/GCGACG | | |
| | 111541174 | (SEQ ID NO: 13) | (SEQ ID NO: 14) | AATCGCC/3IABKFQ | | |
| | | | | (SEQ ID NO: 15, absent labels) | | |
| MAP3K | Chr17: | GTCGTTTGTTTTT | ACTCCTTCCTCTCCGAA | 56-FAM/TGCGGAAGC/ZEN/ | 74 | 60 |
| 14-AS1 | 45262243- | CGTTTTAGGTTC | AACCTCG | GCGAGTTTTATTTTCGA/3IABKFQ | | |
| | 45262339 | (SEQ ID NO: 16) | (SEQ ID NO: 17) | (SEQ ID NO: 18, absent labels) | | |
| SFMBT2 | Chr10: | AATTTCGGACGGATTTT | TTCCCGAATCCCCTTCG | 56-FAM/TTCGAGTTC/ | 70 | 62 |
| | 7410510- | CGTACGGTTC | CCTACG | ZEN/GGAGACGTAGGCGAA | | |
| | 7410579 | (SEQ ID NO: 19) | (SEQ ID NO: 20) | GG/3IABKFQ | | |
| | | | | (SEQ ID NO: 21, absent labels) | | |

TABLE 3
Grid of plasma ctDNA marker results in pre-treatment colorectal cancer case. (1, methylation-positive
(detected at 0.01% or higher); 0, methylation-negative (undetected); control ACTB encodes β-actin.)

| | Collection | Age at | | Stage | mSEPT9 | mSEPT | | | | | |
|---|---|---|---|---|---|---|---|---|---|---|---|
| 54104# | Date | Dx | Location | at Dx | PMR | Interpretation | DNM1P46 | EMBP1 | GATM | VSX2 | Comments |
| 54104-4 | Oct. 3, 2018 | 71 | Colon | IV | 15.91 | 1 | Low | 1 | 1 | 1 | |
| | | | | | | | ACTB | | | | |
| 54104-7 | Sep. 25, 2018 | 63 | Colon | IV | 82.96 | 1 | 1 | 1 | 0 | 1 | |
| 54104-10 | Dec. 5, 2018 | 41 | Colon | IV | 0 | 0 | 0 | 1 | ND | ND | |
| 54104-13 | Oct. 4, 2018 | 65 | Colon | IV | 1.37 | 1 | 1 | 1 | ND | ND | Not done, no |
| | | | | | | | | | | | sample remaining |
| 54104-18 | Mar. 1, 2019 | 66 | Rectal | III | 0 | 0 | Low | 0 | 0 | ND | Not done, no |
| | | | | | | | ACTB | | | | sample remaining |
| 54104-21 | Apr. 8, 2019 | 78 | Colon | IV | 35.36 | 1 | 1 | 1 | 1 | 1 | |
| 54104-33 | Jul. 23, 2019 | 54 | Colon | IV | 507.18 | 1 | 1 | 1 | 0 | 0 | |
| 54104-40 | Aug. 19, 2019 | 80 | Colon | IV | 6883.16 | 1 | 1 | 1 | 1 | 1 | |
| S4104-49 | Oct. 17, 2019 | 56 | Colon | IV | 79.81 | 1 | 1 | 1 | 0 | 0 | |
| 54104-50 | Oct. 7, 2019 | 75 | Colon | IV | 5050.66 | 1 | 1 | 1 | 1 | 1 | |
| 54104-65 | Nov. 27, 2019 | 74 | Colon | II | 81.01 | 1 | 1 | Low | 0 | 1 | |
| | | | | | | | | ACTB | | | |
| 54104-70 | Dec. 16, 2019 | 63 | Colon | IV | 1410.75 | 1 | 1 | 1 | 1 | 1 | |
| 54104-71 | Dec. 18, 2019 | 66 | Colon | IV | 387.21 | 1 | 1 | 1 | 1 | 1 | |
| 54104-77 | Jan. 27, 2020 | 69 | Colon | IV | 0 | 0 | 0 | 0 | 1 | ND | Not done, no |
| | | | | | | | | | | | sample remaining |
| 54104-81 | Feb. 24, 2020 | 84 | Colon | IV | 182.23 | 1 | 1 | 1 | 1 | 1 | |
| 54104-98 | Apr. 1, 2020 | 66 | Colon | III | 236.66 | 1 | 1 | 1 | 0 | 0 | |
| 54104-100 | Apr. 3, 2020 | 65 | Rectal | IV | 136.33 | 1 | 1 | 1 | 0 | 0 | |
| S4104-117 | Jun. 23, 2020 | 51 | Colon | IV | 125.28 | 1 | Failed | Low | 1 | 1 | |
| | | | | | | | | ACTB | | | |
| 54104-121 | Jul. 6, 2020 | 77 | Rectal | III | 0 | 0 | Low | 0 | 0 | 0 | |
| | | | | | | | ACTB | | | | |
| S4104-122 | Jul. 9, 2020 | 64 | Colon | IV | 0 | 0 | Low | 0 | 0 | 0 | |
| | | | | | | | ACTB | | | | |
| 54104-124 | Aug. 3, 2020 | 88 | Colon | IV | 6300 | 1 | 1 | 1 | 1 | 1 | |
| 54104-136 | Sep. 23/2020 | 63 | Rectal | III | 9.45 | 1 | Low | 1 | 1 | 1 | |
| | | | | | | | ACTB | | | | |
| 54104-138 | Sep. 24, 2020 | 56 | Rectal | III | 0 | 0 | Low | 0 | 0 | 0 | |
| | | | | | | | ACTB | | | | |
| 54104-157 | Oct. 27, 2020 | 68 | Colon | IV | 2290 | 1 | 1 | 1 | 1 | 1 | |
| S4104-162 | Nov. 20, 2020 | 73 | Colon | IV | 6565 | 1 | 1 | 1 | 1 | ND | Not done, no |
| | | | | | | | | | | | sample remaining |
| 54104-184 | Dec. 22, 2020 | 63 | Colon | IV | 1668 | 1 | 1 | 1 | 1 | Low | |
| | | | | | | | | | | ACTB | |
| 54104-188 | Dec. 18, 2020 | 63 | Colon | IV | 0 | 0 | Low | 0 | 0 | 0 | |
| | | | | | | | ACTB | | | | |
| 54104-191 | Jan. 6, 2021 | 61 | Colon | III | 4777 | 1 | 1 | 1 | 1 | 1 | |
| 54104-192 | Jan. 6, 2021 | 68 | Rectal | III | 115.63 | 1 | 1 | 1 | 0 | 1 | |
| 54104-194 | Jan. 8, 2021 | 57 | Colon | IV | 0 | 0 | 0 | 0 | 1 | 1 | |
| 54104-218 | Aug. 2, 2021 | 52 | Colon | IV | 0 | 0 | 0 | 0 | 0 | 0 | |
| 54104-224 | Mar. 16, 2021 | 56 | Colon | III | 99.42 | 1 | 1 | 1 | 1 | 1 | |
| 54104-227 | Mar. 30, 2021 | 75 | Colon | IV | 70.2 | 1 | ND | ND | 1 | 1 | Not done, no |
| | | | | | | | | | | | sample remaining |
| 54104-233 | Apr. 5, 2021 | 59 | Rectal | III | 101.84 | 1 | 1 | 1 | 1 | 1 | |
| 54104-251 | May 17, 2021 | 66 | Colon | III | 0 | 0 | 0 | 0 | 0 | 1 | |
| 54104-255 | May 5, 2021 | 57 | Rectal | II | 0.25 | 1 | 1 | 1 | 1 | 0 | |
| 54104-258 | May 24, 2021 | 79 | Rectal | II | 0 | 0 | 0 | 0 | 1 | 1 | |
| 54104-265 | May 18, 2021 | 67 | Colon | II | 0 | 0 | 0 | 0 | 0 | 0 | |
| 54104-272 | May 26, 2021 | 65 | Colon | IV | 112.3 | 1 | 1 | 1 | 0 | 1 | |
| 54104-283 | Jun. 21, 2021 | 66 | Rectal | III | 117.52 | 1 | 0 | 1 | 0 | 0 | |
| 54104-290 | Jun. 30, 2021 | 64 | Rectal | III | 61.1 | 1 | Low | 0 | 0 | 1 | |
| | | | | | | | ACTB, 0 | | | | |
| S4104-296 | Jul. 12, 2021 | 70 | Colon | II | 46.63 | 1 | 1 | 0 | 0 | 0 | |
| 54104-300 | Aug. 17, 2021 | 60 | Colon | I | 0 | 0 | 0 | 0 | 0 | 1 | |
| 54104-307 | Aug. 9, 2021 | 71 | Rectal | III | 120.94 | 1 | 0 | 0 | 0 | 1 | |
| S4104-309 | Aug. 16, 2021 | 52 | Rectal | IV | 25524 | 1 | 1 | 1 | 1 | 1 | |
| 54104-331 | Sep. 8, 2021 | 63 | Colon | II | 107.83 | 1 | 1 | 1 | 1 | 1 | |
| 54104-333 | Sep. 13, 2021 | 54 | Colon | IV | 0 | 0 | 0 | 0 | 0 | ND | Not done, no |
| | | | | | | | | | | | sample remaining |
| 54104-335 | Sep. 22, 2021 | 54 | Rectal | III | 0 | 0 | Low | 0 | 0 | 0 | |
| | | | | | | | ACTB, 0 | | | | |
| 54104-337 | Sep. 17, 2021 | 75 | Colon | III | 0 | 0 | 0 | 0 | 0 | 0 | |
| 54104-347 | Oct. 12, 2021 | 74 | Colon | IV | 47428 | 1 | 1 | 1 | 1 | 1 | |
| | | | | | | 34 | 27 | 29 | 24 | 28 | Total Positive |
| | | | | | | 50 | 39 | 47 | 48 | 43 | Total Valid |
| | | | | | | 68.0 | 69.2 | 61.7 | 50.00 | 65.12 | Sensitivity |
| | | | | | | Reference | 0 | 0 | 3 | 3 | Cases positive |
| | | | | | | | | | | | for which |
| | | | | | | | | | | | mSEPT9 was |
| | | | | | | | | | | | negative |
| | | | | | | | | | | | GATM + VSX2 + |
| | | | | | | | | | | | EMBP1 |
| | | | | | | | | | | | combined >5 |
| | | | | | | | | | | | additional cases |

TABLE 4
Plasma ctDNA marker result grid in pre-surgical colorectal
cancer cases undergoing neoadjuvant chemotherapy treatment.

| SUBJECT # | Cancer | Stage | mSEPT9 | mSEPT9 PMR | DNM1P46 | EMBP1 | SFMBT2 | MAP 3K14-AS1 | VSX2 |
|---|---|---|---|---|---|---|---|---|---|
| Cancer in place | Receiving neoadjuvant therapy | | | | | | | | |
| 54104-10 | Colon | IV | 1 | 27.01 | 1 | 1 | 1 | 1 | 1 |
| 54104-18 | Rectal | III | 1 | 25.65 | 1 | 0 | 1 | 1 | 1 |
| 54104-13 | Colon | IV | 1 | 224.25 | 1 | 1 | 1 | 0 | 1 |
| 54104-103 | Colon | IV | 1 | 465.67 | 1 | 1 | 1 | 1 | Failed |
| 54104-22 | Colon | IV | 1 | 605.1 | 1 | 1 | 0 | 1 | 0 |
| 54104-33 | Colon | IV | 1 | 483.83 | 1 | 1 | 1 | 1 | ND |
| 54104-205 | Rectal | IV | 0 | 0 | 0 | 0 | 0 | 0 | 0 |
| 54104-17 | Rectal | IV | 1 | 360.18 | 1 | 1 | 0 | 1 | 0 |
| 54104-75 | Rectal | IV | 1 | 343.4 | 1 | 1 | 0 | 0 | 1 |
| 54104-4 | Colon | IV | 1 | 23.3 | Low ACTB | ND | 1 | 1 | ND |
| 54104-40 | Colon | IV | 1 | 493.8 | 1 | ND | 1 | 1 | ND |
| 54104-21 | Colon | IV | 1 | 70.44 | 1 | 1 | 1 | 0 | 1 |
| 54104-26 | Colon | IV | 1 | 64.52 | 1 | 1 | 0 | 1 | 1 |
| 54104-28 | Colon | IV | 1 | 49.13 | 1 | 1 | 1 | 0 | ND |
| 54104-125 | Rectal | IV | 0 | 0 | 0 | 0 | 0 | 0 | 1 |
| 54104-115 | Colon | IV | 0 | 0 | Failed | 0 | 0 | 0 | 0 |
| 54104-136 | Rectal | III | 0 | 0 | Low ACTB | 0 | 0 | 0 | 1 |
| 54104-127 | Rectal | III | 0 | 0 | ND | ND | ND | 1 | ND |

Healthy Controls

| | | | | | | | | | |
|---|---|---|---|---|---|---|---|---|---|
| 54104-141 | NA | NA | 1 | 0.26 | 0 | 0 | 0 | 0 | 0 |
| 54104-8 | NA | NA | 0 | 0 | 0 | 0 | 0 | 0 | 0 |
| 54104-185 | NA | NA | 0 | 0 | 0 | 0 | 0 | 0 | 0 |
| 54104-211 | NA | NA | 0 | 0 | Failed | 0 | 0 | 0 | ND |
| 54104-223 | NA | NA | 0 | 0 | 0 | 0 | 0 | 0 | 0 |
| 541-4-96 | NA | NA | 0 | 0 | 0 | 0 | 0 | 0 | 0 |

TABLE 5
Grid of selected markers used to detect minimal residual disease in patients
following surgical resection who later recurred. 1, ctDNA detected. 0, ctDNA
not detected. PMR, percentage of methylated reference value as a measure of
ctDNA levels detected by each methylated ctDNA marker. Signatera test is a
comparator, performed via next-generation sequencing of known tumor genetic
mutations in cfDNA, MTM/mL, mutant tumor molecules per mL plasma detected.

| | | GATM | | VSX2 | | mSEPT9 | | Signatera |
|---|---|---|---|---|---|---|---|---|
| Patient | GATM | PMR | VSX2 | PMR | mSEPT9 | PMR | Signatera | MTM/mL |
| 54104-62 | 1 | 0.28 | 0 | 0 | 0 | 0 | 1 | 1.49 |
| 54104-66 | 1 | 6.93 | 1 | 3.59 | 1 | 167.46 | 1 | 155.7 |
| 54104-137 | 1 | 5.33 | 1 | 0.76 | 1 | 681.73 | 1 | 28.39 |
| 54104-323 | Low | NA | 1 | 18.69 | 0 | 0 | 1 | 10.41 |
| | ACTB | | | | | | | |
| 54104-329 | 1 | 52.12 | Low | NA | 1 | 432.32 | 1 | 8.92 |
| | | | ACTB | | | | | |
| 54104-338 | 1 | 0.77 | 1 | 0.01 | 1 | 8.49 | 0 | 0 |

TABLE 6
Grid of methylation status in 36 stage I/II colorectal cancer tumor tissues in the Discovery series. 1, methylation-
positive (methylation level >25%); 0, methylation-negative (methylation level 0-25%). Bolded text shows three “problem-
maker” tumors that were negative for all markers (possibly due to low proportion of tumor cells in the sample).

| | 1 | 2 | 3 | 4 | 5 | 6 | 7 | 8 | 9 | 10 | 11 |
|---|---|---|---|---|---|---|---|---|---|---|---|
| | T\|CRC173 | T\|CRC188 | T\|CRC18 | T\|CRC187 | T\|CRC16 | T\|CRC199 | T\|CRC227 | T\|CRC240 | T\|CRC241 | T\|CRC242 | T\|CRC245 |
| SEPTIN9 | 0 | 1 | 0 | 0 | 0 | 1 | 0 | 0 | 0 | 1 | 1 |
| GATM | 1 | 1 | 0 | 1 | 0 | 0 | 0 | 0 | 1 | 1 | 0 |
| DNM1P46 | 1 | 1 | 0 | 1 | 0 | 1 | 1 | 0 | 1 | 1 | 1 |
| EMBP1 | 1 | 1 | 0 | 1 | 0 | 1 | 0 | 1 | 1 | 1 | 1 |
| VSX2 | 1 | 1 | 0 | 1 | 0 | 1 | 1 | 1 | 1 | 1 | 1 |
| MAP3K14- | 0 | 1 | 0 | 1 | 0 | 1 | 1 | 1 | 1 | 1 | 0 |
| AS1 | | | | | | | | | | | |
| TOTAL | 1 | 1 | 0 | 1 | 0 | 1 | 1 | 1 | 1 | 1 | 1 |
| | 12 | 13 | 14 | 15 | 16 | 17 | 18 | 19 | 20 | 21 | 22 |
| | T\|CRC249 | T\|CRC326 | T\|CRC335 | T\|CRC39 | T\|CRC392 | T\|CRC393 | T\|CRC405 | T\|CRC409 | T\|CRC429 | T\|CRC430 | T\|CRC431 |
| SEPTIN9 | 0 | 0 | 1 | 0 | 0 | 1 | 1 | 1 | 0 | 0 | 0 |
| GATM | 1 | 1 | 0 | 0 | 0 | 1 | 0 | 1 | 1 | 1 | 1 |
| DNM1P46 | 1 | 1 | 0 | 0 | 1 | 1 | 1 | 1 | 1 | 1 | 0 |
| EMBP1 | 1 | 1 | 1 | 0 | 0 | 1 | 1 | 1 | 1 | 1 | 0 |
| VSX2 | 1 | 1 | 1 | 0 | 0 | 1 | 1 | 1 | 0 | 1 | 0 |
| MAP3K14- | 1 | 1 | 1 | 0 | 1 | 1 | 0 | 0 | 0 | 1 | 1 |
| AS1 | | | | | | | | | | | |
| TOTAL | 1 | 1 | 1 | 0 | 1 | 1 | 1 | 1 | 1 | 1 | 1 |
| | 23 | 24 | 25 | 26 | 27 | 28 | 29 | 30 | 31 | 32 | 33 |
| | T\|CRC431 | T\|CRC433 | T\|CRC442 | T\|CRC451 | T\|CRC465 | T\|CRC483 | T\|CRC494 | T\|CRC639 | T\|CRC735 | T\|CRC744 | T\|CRC832 |
| SEPTIN9 | 1 | 0 | 1 | 1 | 0 | 1 | 0 | 1 | 0 | 0 | 1 |
| GATM | 1 | 0 | 1 | 1 | 0 | 1 | 0 | 1 | 1 | 1 | 1 |
| DNM1P46 | 1 | 0 | 0 | 1 | 1 | 1 | 1 | 0 | 1 | 1 | 1 |
| EMBP1 | 1 | 1 | 1 | 1 | 0 | 1 | 1 | 1 | 1 | 1 | 1 |
| VSX2 | 1 | 0 | 0 | 1 | 0 | 1 | 1 | 0 | 1 | 1 | 1 |
| MAP3K14- | 1 | 0 | 1 | 1 | 0 | 1 | 0 | 1 | 1 | 1 | 1 |
| AS1 | | | | | | | | | | | |
| TOTAL | 1 | 1 | 1 | 1 | 1 | 1 | 1 | 1 | 1 | 1 | 1 |

| | | | | | | | prevalence |
|---|---|---|---|---|---|---|---|
| | | | | | | | w/o |
| | 34 | 35 | 36 | | | #problem | problem |
| | T\|CRC834 | T\|CRC952 | T\|CRC977 | #positive | prevalence | maker | makers |
| SEPTIN9 | 1 | 0 | 1 | 16 | 44.44% | 3 | 48.48% |
| GATM | 1 | 1 | 1 | 22 | 61.11% | 3 | 66.67% |
| DNM1P46 | 1 | 1 | 1 | 27 | 75.00% | 3 | 81.82% |
| EMBP1 | 1 | 1 | 1 | 29 | 80.56% | 3 | 87.88% |
| VSX2 | 1 | 1 | 1 | 26 | 72.22% | 3 | 78.79% |
| MAP3K14- | 0 | 0 | 0 | 22 | 61.11% | 3 | 66.67% |
| AS1 | | | | | | | |
| TOTAL | 1 | 1 | 1 | 33 | 91.67% | 3 | 100.00% |

1. A method for methylation analysis of one or more marker genetic loci in a subject in need thereof, comprising:
measuring or requesting measurement of a methylation level of the one or more marker genetic loci in a biological sample obtained from the subject, said one or more marker genetic loci comprising gene of: DNM1P46, EMBP1, GATM, VSX2, MAP3K14-AS1, LAYN, SFMBT2, or a combination thereof,
wherein the subject has colorectal cancer or requests determination regarding colorectal cancer.

2. The method of claim 1, further comprising measuring a methylation level of SEPT9.

3. The method of claim 1, wherein a higher methylation level is measured for each of the measured one or more marker genetic loci relative to respective reference methylation level.

4. A method of screening for colon cancer in a subject or treating the subject, comprising:
performing colonoscopy on, or providing a treatment to, a subject measured in a biological sample of the subject with a methylation level of one or more marker genetic loci above respective reference methylation level,
wherein the one or more marker genetic loci comprise gene of: DNM1P46, EMBP1, GATM, VSX2, MAP3K14-AS1, LAYN, SFMBT2, or a combination thereof, and optionally wherein the subject is also measured with a methylation level of SEPT9 in the biological sample above respective reference methylation level.

5. A method for treating a subject with colorectal cancer, comprising:
performing methylation analysis for the subject according to the method of claim 1, wherein the one or more marker genetic loci comprise gene of: DNM1P46, EMBP1, GATM, VSX2, MAP3K14-AS1, LAYN, SFMBT2, or a combination thereof, and optionally further measuring or requesting measuring of a methylation level of SEPT9, and
providing a treatment to the subject if the methylation level of the one or more marker genetic loci in the biological sample is above respective reference methylation level.

6. A method of assaying a subject having undergone surgery or a treatment against colorectal cancer, identifying presence or absence of minimal residual disease in the subject, and/or identifying risk of cancer recurrence or relapse in the subject, comprising:
performing methylation analysis for the subject according to the method of claim 1, said one or more marker genetic loci comprising gene of: DNM1P46, EMBP1, GATM, VSX2, MAP3K14-AS1, LAYN, or SFMBT2, or a combination thereof, optionally the one or more marker genetic loci further comprising SEPT9;
wherein a higher methylation level of the one or more measured maker genetic loci relative to respective reference methylation levels indicates presence of the minimal residual disease and/or risk of cancer recurrence or relapse in the subject.

7. (canceled)

8. (canceled)

9. (canceled)

10. (canceled)

11. The method of claim 1, wherein the one or more marker genetic loci if measured comprises genomic coordinates of:
chr1:121519493-121519559 for EMBP1,
chr15:99806584-99806722 for DNM1P46,
chr15:45378270-45378365 for GATM,
chr14:45378270-74240641 for VSX2,
chr11:111540870-111541174 for LAYN,
chr17:45262243-45262339 for MAP3K14-AS1, and
chr10:7410510-7410579 for SFMBT2.

12. The method of claim 1, wherein the biological sample comprises cell-free DNA (cfDNA).

13. The method of claim 12, wherein the cfDNA comprises circulating tumor DNA (ctDNA).

14. The method of claim 1, wherein the biological sample comprises colorectal mucosa or is obtained from the colorectal mucosa of the subject.

15. The method of claim 1, wherein the biological sample comprises plasma or blood, or is obtained from the subject's plasma or blood.

16. The method of claim 1, wherein the biological sample is obtained from the subject's feces.

17. The method of claim 1, wherein the biological sample comprises tumor tissue or is a biopsy obtained from a cancerous tissue of the subject.

18. The method of claim 1, wherein the subject is a human subject with a stage I or II colon cancer or stage I or II rectal cancer.

19. The method of claim 1, wherein the subject is a human subject with a stage III or IV colon cancer or stage III or IV rectal cancer.

20. The method of claim 3, wherein the respective reference methylation level is measured in normal colon mucosa (NCM) of the subject.

21. The method of claim 3, wherein the respective methylation level is measured in a control subject free of colorectal cancer or another cancer.

22. (canceled)

23. The method of claim 1, wherein the one or more marker genetic loci measured comprise MAP3K14-AS1, DNM1P46, EMBP1, GATM, and VSX2, and optionally further comprising SEPT9.

24. A method of assessing efficacy or effectiveness of a treatment to a subject with colorectal cancer, or monitoring progression of the colorectal cancer in the subject, comprising:
measuring a methylation level of one or more marker genetic loci in a first biological sample obtained from the subject at a time t0,
measuring a methylation level of the one or more marker genetic loci in a second biological sample obtained from the subject at a time t1, said time t1 being subsequent to said time t0, and for assessing the efficacy or effectiveness of the treatment said time t1 being subsequent to the treatment,
wherein the one or more marker genetic loci comprise gene of: DNM1P46, EMBP1, GATM, VSX2, MAP3K14-AS1, LAYN, or SFMBT2, or a combination thereof, and optionally further comprising SEPT9, and
wherein the treatment is indicated to be effective, or the colorectal cancer is indicated to show regression or has not worsened, when DNM1P46, EMBP1, GATM, VSX2, MAP3K14-AS1, LAYN, SFMBT2, and SEPT9, if selected, each has a lower methylation level in the second biological sample obtained at the time t1 relative to that in the first biological sample obtained at the time t0; and
wherein measuring the methylation level comprises:
a) treating DNA in the biological sample with one or more reagents to convert unmethylated cytosine bases to uracil sulfonate or another base having a different binding behavior than cytosine, while methylated cytosine bases remain unchanged;
b) amplifying the treated DNA in the presence of a forward primer oligonucleotide and a reverse primer oligonucleotide, and optionally a polymerase, wherein each of the forward primer oligonucleotide and the reverse primer oligonucleotide hybridizes specifically onto the treated DNA of the one or more marker genetic loci; and
c) sequencing the amplified DNA in step b) to deduce percentage of methylated cytosine bases in the one or more marker genetic loci as the methylation level, wherein the amplified DNA in step b) is fewer than 80 bp in length, optionally 50-54, 55-60, 61-65, 66-70, or 71-74 bp in length.

25. (canceled)

26. (canceled)

27. A kit, comprising:
a first oligonucleotide which hybridizes onto a first region of one or more marker genetic loci selected from genes of: DNM1P46, EMBP1, GATM, VSX2, MAP3K14-AS1, LAYN, SFMBT2, a combination of DNM1P46, EMBP1, GATM, VSX2, MAP3K14-AS1, LAYN, and SFMBT2, and a combination of SEPT9 and one or more of DNM1P46, EMBP1, GATM, VSX2, MAP3K14-AS1, LAYN, and SFMBT2;
a second oligonucleotide which hybridizes onto a second region of each of the selected one or more marker genetic loci; and
optionally a third oligonucleotide which hybridizes onto a third region of each of the one or more marker genetic loci, wherein the third oligonucleotide is modified with a detectably labeled moiety, a quencher, or both, and wherein the third region includes at least three CpG dinucleotide sites; and
optionally a polymerase;
wherein the first and the second oligonucleotides are each 22-30 bases in length, and each of the first region and the second region comprises at least one CpG dinucleotide site; and
wherein optionally the first and the second oligonucleotides are selected from a forward PCR primer sequence and a corresponding reverse PCR primer sequence of Table 2, and the third oligonucleotide is selected from a Probe sequence of Table 2.

28. (canceled)
